(19) United States
(12) Patent Application Publication
(10) Pub. No.: US 2025/0301998 A1
(43) Pub. Date: Oct. 2, 2025

(54) PURPLE-FLESHED SWEETPOTATO LINES AND METHODS THEREOF

(71) Applicant: The Texas A&M University System, College Station, TX (US)

(72) Inventors: Ming Gao, Cypress, TX (US); Willie DeWalt, Prairie View, TX (US)

(21) Appl. No.: 19/086,700

(22) Filed: Mar. 21, 2025

Related U.S. Application Data
(60) Provisional application No. 63/571,066, filed on Mar. 28, 2024.

(51) Int. Cl. A01H 6/82; A01H 5/06

(52) U.S. Cl. CPC A01H 6/82; A01H 5/06

(57) ABSTRACT

The invention provides plants of the sweetpotato lines designated ‘P4’ and ‘R5’. The invention thus relates to the plants, cells, plant parts, and tissue cultures of the lines ‘P4’ and ‘R5’, and to methods for producing a sweetpotato plant produced by crossing a sweetpotato plant of line ‘P4’ or ‘R5’ with another sweetpotato plant, such as a plant of another line. The invention further relates to Ipomoea batatas seeds and plants produced by crossing plants of line ‘P4’ or ‘R5’ with plants of another line. The invention further relates to the genetic complements and hybrid genetic complements of plants of lines ‘P4’ and ‘R5’.

### CROSS-REFERENCE TO RELATED APPLICATIONS

This application claims the benefit of U.S. Provisional Application No. 63/571,066, filed Mar. 28, 2024, herein incorporated by reference in its entirety.

### STATEMENT OF GOVERNMENT RIGHTS

This invention was made with government support under Grant Nos. 2014-38821-22429 and 2021-38821-34577 awarded by the USDA National Institute of Food and Agriculture. The government has certain rights in the invention.

### FIELD OF THE INVENTION

The present invention relates to the field of plant breeding and, more specifically, to Ipomoea batatas plants comprising purple-flesh and improved weevil-resistance. In particular, the invention relates to plants of the sweetpotato lines designated ‘P4’ and ‘R5’, and derivatives and tissue cultures thereof.

### DESCRIPTION OF RELATED ART

Ipomoea batatas, commonly known as sweetpotato, is a dicotyledonous plant belonging to the Convolvulaceae family. Sweetpotatoes, the storage root of the sweetpotato plant, have special nutrition values and health benefits, as they contain abundant vitamins (e.g. ß-carotene, Vitamin B1 and C), antioxidant micronutrients, minerals, dietary fibers, and one of the few non-fat sources of Vitamin E. See, e.g., Woolfe, J. Sweetpotato—A versatile and nutritious food for all. In Product Development for Root and Tuber Crops, G. J. Scott and P. I. Ferguson and J. E. Herrera, eds. (1993) pp. 221-232. The storage root has various shapes, including ellipsoid, fusiform, or elongated shapes, and colors, such as red, yellow, brown, white, and purple. The plant is generally cultivated as an annual crop and harvested after one growing season.

The sweetpotato plant is traditionally propagated by vegetative propagation using vine/stem cuttings or tubers. The sprouts produced from the tuber are excellent planting material for the vegetative propagation of sweet potato. Biotechnological approaches using different plant tissue culture techniques have also been developed for producing a large number of true-to-type plants. In particular, plant tissue culture is an efficient and reliable method for the large-scale production of high-quality and disease-free plants in a short time period. Biotechnological advances through plant tissue culture also have the potential to provide new prospects for the improvement of sweet potato with high nutritional and pharmaceutical value. In vitro plant regeneration protocols for sweetpotato include meristem culture, callus culture, direct adventitious organogenesis, somatic embryogenesis, and synthetic seed technology. See, for example, Behera et. al (Biology and biotechnological aspect of sweet potato (Ipomoea batatas L.): a commercially important tuber crop. Planta. 256:40 (2022)).

Although sweetpotato is generally regarded as highly self-incompatible, there are differences within the species with respect to self- and cross-incompatibility. The effects of incompatibility in the breeding of sweetpotato, the various types of incompatibilities, methods to determine and distinguish incompatibility and sterility, and various techniques that can be used to overcome incompatibility to enhance the genetic improvement of the crop, offer models for the development and maintenance of Ipomoea batatas lines. See, for example, Gurmu et. al (Self- and cross-incompatibilities in sweetpotato and their implications on breeding. Australian Journal of Crop Science. 13. 2074-2078. (2013)).

Among the various types of sweetpotatoes, purple-fleshed sweetpotatoes are especially beneficial to our health, owing to their high anthocyanin content. Anthocyanin is the pigment responsible for the brilliant purple flesh of these sweetpotatoes, and is also an excellent antioxidant, and has been demonstrated to possess many preventative health benefits (Montilla et al., Anthocyanins in Purple Sweet Potato (Ipomoea batatas L.) Varieties. Fruit, Vegetable and Cereal Science and Biotechnology. 2011 5:19-24; Naseri et al., Anthocyanins in the Management of Metabolic Syndrome: A Pharmacological and Biopharmaceutical Review. Front Pharmacol. 2018. 9:1310. 10.3389/fphar.2018.01310), particularly related to reducing risks of cardiovascular disease, cancer, and the metabolic syndrome. The anthocyanin contents in various purple-fleshed sweetpotatoes may vary widely, e.g. 558-2477 mg/100 g DM (Dry Matter) (Gras et al., Anthocyanins from purple sweet potato (Ipomoea batatas (L.) Lam.) and their color modulation by the addition of phenolic acids and food-grade phenolic plant extracts. Food Chem. 2017. 235:265-274. 10.1016/j.foodchem.2017.04.169), but are generally much higher than those in other vegetables and fruits.

In recent years, the market demand for purple-fleshed sweetpotatoes have been steadily expanding, and the market price for purple-fleshed sweetpotatoes is typically more than double that of orange-fleshed sweetpotatoes. However, production of sweetpotatoes in the southern United States has been declining due in part to a severe weevil infestation in this region. In particular, Texas was the third largest sweetpotato producer in 2000, but produces less than 1% of the national total output as of 2017. In order to make sweetpotato an economically viable crop in these regions, it is crucial to develop sweetpotato varieties with improved weevil resistance and agronomic characteristics meeting or exceeding market trends. For example, purple-fleshed sweetpotatoes that are currently cultivated in the United States all have dryer and tougher mouthfeel when cooked as compared to popular orange-fleshed sweetpotatoes. Developing novel purple-fleshed sweetpotatoes that not only have improved weevil resistance, but also produce sweetpotatoes with improved texture is highly desirable. Thus, a continuing need exists in the art to develop new purple-fleshed sweetpotato varieties with improved resistance to weevil infection as well as more favorable texture.

### SUMMARY OF THE INVENTION

In one aspect, provided herein is a sweetpotato plant of line ‘P4’. In another aspect, provided herein is a sweetpotato plant of line ‘R5’. Also provided are sweetpotato plants having all the physiological and morphological characteristics of such a plant. Parts of said sweetpotato plants of the present invention are also provided, including e.g. a flower, pollen, a leaf, an ovule, an embryo, a cutting, an axillary bud, a stem, a root, or a seed of the respective plants. In other embodiments, a tissue culture of regenerable cells of a sweetpotato plant of line ‘P4’ or of line ‘R5’ are also provided. The tissue culture will preferably be capable of regenerating sweetpotato plants capable of expressing all of the physiological and morphological characteristics of the starting plant, and of regenerating plants having substantially the same genotype as the starting plant. Examples of some of the physiological and morphological characteristics of the line ‘P4’ and the line ‘R5’ include those traits set forth herein, respectively. The regenerable cells in such tissue cultures may be derived, for example, from embryos, meristems, pollen, leaves, anthers, roots, root tips, pistils, and flowers. In further embodiments, a sweetpotato plant regenerated from the tissue culture, wherein the regenerated plant comprises all of the physiological and morphological characteristics of a sweetpotato plant of line ‘P4’ or ‘R5’ is provided.

In another aspect, a method for producing a first generation progeny sweetpotato seed is provided, wherein the method comprises the steps of: crossing the plant of claim 1 or 2 with itself or a second sweetpotato plant and harvesting the resultant sweetpotato seed. In some embodiments, the method further comprises crossing a sweetpotato plant of line ‘P4’ or ‘R5’ with itself or a second sweetpotato plant and harvesting the resultant sweetpotato seed. In certain embodiments, the second sweetpotato plant is a plant of sweetpotato line ‘P4’. In further embodiments, the second sweetpotato plant is a plant of sweetpotato line ‘R5’. Also provided is a first generation progeny sweetpotato seed produced by such method, wherein the first generation progeny sweetpotato plant produced from said seed has all of the physiological and morphological characteristics of a plant of sweetpotato line ‘P4’ or ‘R5’. In still further embodiments, provided herein are sweetpotato plants produced by growing the first generation progeny sweetpotato seed.

In another aspect of the invention, a method of vegetatively propagating a sweetpotato plant of line ‘P4’ or ‘R5’ is provided, wherein the method comprises collecting tissue capable of being propagated from the plant of claim 1 or 2; and propagating a plant from said tissue.

In yet another aspect of the invention, methods of introducing a trait into a sweetpotato plant are provided. In one embodiment, the method comprises comprises introducing a transgene or a single locus conversion into a sweetpotato plant of line ‘P4’ or ‘R5’. In certain embodiments, said sweetpotato plant comprises the transgene or the single locus conversion and otherwise comprises all of the physiological and morphological characteristics of a plant of sweetpotato line ‘P4’ or ‘R5’. In specific embodiments, the transgene or single locus comprises a nucleic acid sequence that enables site-specific genetic recombination or confers a trait selected from the group consisting of male sterility, herbicide tolerance, insect resistance, pest resistance, disease resistance, improved digestibility, improved energy content, improved forage or seed yield, improved winterhardiness, improved nitrogen fixation, modified fatty acid metabolism, abiotic stress resistance, flowering time, altered seed amino acid composition, and modified carbohydrate metabolism.

In another aspect, a method of introducing a single-locus conversion into a sweetpotato plant of line ‘P4’ or ‘R5’ is provided, wherein the method comprises crossing a sweetpotato plant of line ‘P4’ or ‘R5’ with a second sweetpotato plant to produce a first generation of progeny plants, wherein the second sweetpotato plant comprises the single locus; and selecting a progeny plant that comprises the single locus. In yet another aspect, provided herein is a method of for introducing a transgene or a single locus conversion into a population of sweetpotato plants, the method comprising the steps of: (a) modifying a sweetpotato plant of line ‘P4’ or ‘R5’ by introducing a transgene or a single locus conversion; and (c) crossing the modified sweetpotato plant of step (a) with a population of sweetpotato plants to produce a population of progeny plants, wherein at least a progeny plant comprises the transgene or single locus conversion. In some embodiments, the method further comprises applying a selection technique to the population produced in step (b) to select said progeny plants that comprise the transgene or single locus conversion.

In a further aspect, a method of producing a commodity plant product is provided, the method comprising producing a commodity plant product from a sweetpotato plant of line ‘P4’ or ‘R5’ or plant part thereof. In certain embodiments, the commodity plant product is selected from a group consisting of tuberous root, flour, starch, juice, bread, and pectin. In other embodiments, the commodity plant product comprises at least one cell of sweetpotato line ‘P4’ or ‘R5’.

In another aspect, provided herein is a method of plant breeding comprising applying plant breeding techniques to a sweetpotato plant of line ‘P4’ or ‘R5’. In some embodiments, the method comprises producing a sweetpotato line ‘P4’-derived sweetpotato plant; or a sweetpotato line ‘R5’-derived sweetpotato plant. In specific embodiments, the plant breeding techniques comprise recurrent selection, mass selection, hybridization, open-pollination, backcrossing, modified backcrossing, pedigree breeding, mutation breeding, or marker assisted selection. In further embodiments, the method comprises selecting a sweetpotato line ‘P4’-derived sweetpotato plant that comprises: a purple skin trait found in sweetpotato line ‘P4’; a purple flesh trait found in sweetpotato line ‘P4’; or a weevil resistance trait found in sweetpotato line ‘P4’. In other embodiments, the method comprises selecting a sweetpotato line ‘R5’-derived sweetpotato plant that comprises: a self-fertility trait found in sweetpotato line ‘R5’; a purple flesh trait found in sweetpotato line ‘R5’; or a weevil resistance trait found in sweetpotato line ‘R5’.

### BRIEF DESCRIPTION OF THE DRAWINGS

FIG. 1: Illustrates the morphological and physiological characteristics of sweetpotato lines designated ‘P4’ and ‘R5’, including a closeup view of the growing plants (Panel A); freshly harvested storage sweetpotato (Panel B); and the skin and flesh of cured sweetpotatos from lines designated ‘P4’ and ‘R5’ (Panel C).

FIG. 2: Shows the average fresh sweetpotato yields of lines ‘P4’ and ‘R5’ as compared to parental cultivar, ‘Resisto’, grown side-by-side under two fertilizer regimens (NPK1 and NPK2).

FIG. 3: Summary of hybridization and selection processes for sweetpotato lines ‘P4’ and ‘R5’. Reciprocal hybridizations between the two outbred parental lines were performed using natural open-field pollination over three growing seasons. Seeds were collected from each of the two lines as a maternal line. The numbers of germinated and phenotypically evaluated lines from each maternal line are listed.

### DETAILED DESCRIPTION OF THE INVENTION

### Definitions

Allele: Any of one or more alternative forms of a gene locus, all of which alleles relate to one trait or characteristic. In a diploid cell or organism, the two alleles of a given gene occupy corresponding loci on a pair of homologous chromosomes.

Backcrossing: A process in which a breeder repeatedly crosses hybrid progeny back to one of the parents, for example, a first generation hybrid (F₁) with one of the parental genotypes of the F₁ hybrid.

Crossing: The pollination of a female flower of an Ipomoea batatas plant (i.e. sweetpotato plant), thereby resulting in the production of seed from the flower.

Cross-pollination: Fertilization by the union of two gametes from different plants.

F₁ Hybrid: The first generation progeny of the cross of two plants.

Genetic Complement: An aggregate of nucleotide sequences, the expression of which sequences defines the phenotype in Ipomoea batatas plants, or components of plants including cells or tissue.

Genotype: The genetic constitution of a cell or organism.

Haploid: A cell or organism having one set of the two sets of chromosomes in a diploid.

Linkage: A phenomenon wherein alleles on the same chromosome tend to segregate together more often than expected by chance if their transmission was independent.

Marker: A readily detectable phenotype, preferably inherited in codominant fashion (both alleles at a locus in a diploid heterozygote are readily detectable), with no environmental variance component, i.e., heritability of 1.

Non-transgenic mutation: A mutation that is naturally occurring (spontaneous), or induced by conventional methods (e.g. exposure of plants to radiation or mutagenic compounds), not including mutations made using recombinant DNA techniques.

Phenotype: The detectable characteristics of a cell or organism in which the characteristics are the manifestation of gene expression.

Quantitative Trait Loci (QTL): Genetic loci that contribute, at least in part, certain numerically representable traits that are usually continuously distributed.

Regeneration: The development of a plant from tissue culture.

SSR profile: A profile of simple sequence repeats used as genetic markers and scored by gel electrophoresis following PCR amplification using flanking oligonucleotide primers.

Self-pollination: The transfer of pollen from the anther to the stigma of the same plant.

Single Locus Converted (Conversion) Plant: Plants that are developed by a plant breeding technique called backcrossing or by genetic engineering of a locus, wherein essentially all of the morphological and physiological characteristics of a plant are recovered in addition to the characteristics conferred by the single locus transferred into the plant via the backcrossing or genetic engineering technique.

Substantially Equivalent: A characteristic that, when compared, does not show a statistically significant difference (e.g., p=0.05) from the mean.

Tissue Culture: A composition comprising isolated cells of the same or a different type or a collection of such cells organized into parts of a plant.

Transgene: A genetic sequence that has been introduced into the nuclear or chloroplast genome of an Ipomoea batatas plant by genetic transformation or site-specific modification.

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice.

However, those of skill in the art should, in light of the present disclosure, appreciate that many changes can be made in the specific embodiments that are disclosed and still obtain a like or similar result without departing from the spirit and scope of the invention.

### Sweetpotato Line ‘P4’

The disclosure provides Ipomoea batatas plants having purple-flesh and improved weevil-resistance.

### A. Origin and Breeding History

Sweetpotato line ‘P4’ was selected from among 402 germinated hybrids from a ‘CH-Purple’-mothered x ‘Resisto’ cross. During trials, plants of the selected line were surprisingly observed to found that the ‘P4’ line displayed improved weevil resistance, and yielded sweetpotatoes having features for processing and vegetables uses. For example, the ‘P4’ line has a very robust growing pattern, improved weevil resistance, and average disease resistance, and produces sweetpotatoes in good shape and sizes (if not overgrown). It has a day-neutral photoperiod, producing copious flowers under cool temperature in pots and in field. It has a wider cross-compatibility, capable of crossing to the parental lines, many F1 siblings and many germplasm lines in the breeding program. In particular, the young leaves and shoots of the ‘P4’ line contains a high anthocyanin content as indicated by the purple color of young leaves, and not as stingy and bitter when cooked as most of the breeding lines and cultivars used in the breeding program. Additionally, the ‘P4’ line produced sweetpotatoes having much less reduction of anthocyanin content as compared to those from the maternal ‘CH-Purple’ line and the other purple-fleshed hybrids evaluated in during the breeding process, and a high dry-matter content. This combination of traits is unique compared to all other known sweetpotato varieties.

The male parent, ‘Resisto’ is characterized by its high yield and resistance to disease and insect pests. The female parent, ‘CH-Purple’, was originally sprouted from sweetpotatoes sold at the Waller County Farmers Market in Hempstead, TX. ‘CH-Purple’ is characterized by its purple flesh and reddish-purple skin.

Asexual reproduction of sweetpotato line ‘P4’ by sprouted plantlets or slips since May of 2016 has demonstrated that sweetpotato line ‘P4’ reproduces true-to-type for all of the physiological and morphological characteristics described herein. All of the physiological and morphological characteristics described herein are firmly fixed and retained through successive generations of such asexual propagation.

### B. Phenotypic Description

In accordance with another aspect of the present invention, there is provided a sweetpotato plant having the morphological characteristics of sweetpotato line ‘P4’. A description of the morphological and physiological characteristics of sweetpotato line ‘P4’ is presented below.

The following characteristics have been repeatedly observed and can be used to distinguish ‘P4’ as a new and distinct variety of Ipomoea batatas plant:

TABLE 1
Summary of Morphological and Physiological Characteristics of Sweetpotato line ‘P4’

| Characteristics | ‘P4’ |
|---|---|
| Leaves | Large to very large leaves with entire to 3 lobs, purple-young leaves, but |
| | deep-green adult leaves. |
| Vines | Very long, mostly purple-colored, and large-diametered vines, and very brittle |
| | young vines. |
| Photoperiod and | Day-neutral photoperiod, copious flowers under cool temperature. |
| Flowers | |
| Cross-compatibility | Self-incompatible, and cross-compatible to both parental lines and many |
| | siblings including ‘R5’. |
| Roots (fibrous) | Purple skin |
| Storage Roots | Purple to pinkish skin with fine longitudinal strips; purple flesh with spotty |
| | orange to whitish colors; 7 to 15 relative short-round sweetpotatoes per plant |
| | and many jumbo-sized ones if grown over 120 days after planting. |
| Weevil Infection | 50 to 60% in non-treated but crop-rotated plots; and <10% in insecticide- |
| | treated plots. |
| Resistance Against | Susceptible to the southern potato wireworm (Conoderus falli Lane) and |
| Other Soil Insects | white grubs. |
| Leaf-eater Resistance | Poor |
| Virus Resistance | Highly resistant against SPLCV |
| Other Notable | Susceptible to fusarium wilt, but somewhat resistant against stem rot |
| Disease Resistance | |

TABLE 2
International Board for Plant Genetic Resources (IBPGR) Sweetpotato Descriptors for Sweetpotato
line ‘P4’. (See Genesys (2018). Descriptors for sweet potato. Version 1991)

| Descriptor | | Coded | Title for | Additional | |
|---|---|---|---|---|---|
| Title | Description | Value | Coded Value | Description | |
| Twining | Ability of vines to climb | 0 | No-Twining | | |
| | adjacent stakes placed in those | | | | |
| | accessions showing twining | | | | |
| | characters | | | | |
| Plant type | Length of the main vines | 9 | Extremely | >250 | cm |
| | | | spreading | | |
| Ground | Estimated percentage of the | 9 | Total | >90% | |
| cover | ground cover recorded 35-40 | | | | |
| | days after planting | | | | |
| Vine | Average length of at least | 3 | Short | 3-5 | cm |
| internode | three internodes located in the | | | | |
| length | middle section of the vine. | | | | |
| Vine | Average diameter of at least | 5 | Intermediate | 7-9 | mm |
| internode | three internodes located in the | | | | |
| diameter | middle section of the vine. | | | | |
| Predominant | Anthocyanine (purple | 4 | Green with | | |
| vine colour | pigmentation) present in the | | many purple | | |
| | vines beside the green colour. | | spots | | |
| | The predominant colour | | | | |
| | should be evaluated | | | | |
| | considering the whole vine | | | | |
| | from base to tip. | | | | |
| Secondary | Anthocyanine (purple | 4 | Purple base | | |
| vine colour | pigmentation) present in the | 5 | Purple tip | | |
| | vines beside the green colour. | 6 | Purple nodes | | |
| | The predominant colour | | | | |
| | should be evaluated | | | | |
| | considering the whole vine | | | | |
| | from base to tip. The | | | | |
| | secondary colour is more | | | | |
| | easily evaluated using | | | | |
| | younger vines. | | | | |
| Vine tip | Degree of hairiness of | 0 | Absent | | |
| pubescence | immature leaves recorded at | | | | |
| | the apex of the vines | | | | |
| General | Described from mature leaves | 3 | Cordate | Kidney-shaped | |
| outline of | located in the middle section | 4 | Triangular | Heart-shaped | |
| the leaf | of the vine. | 5 | Hastate | Trilobular and spear- | |
| | | 6 | Lobed | shaped, with the basal | |
| | | | | lobes more or less | |
| Leaf lobes | | 1 | Very slight | Teeth | |
| type | | 3 | Slight | | |
| | | 5 | Moderate | | |
| Leaf lobe | Most leaves of sweet potato | 1 to 3 | | | |
| number | have two basal lobes and they | | | | |
| | should not be counted. Record | | | | |
| | the predominant number of | | | | |
| | the lateral and central leaf | | | | |
| | lobes observed on the leaves | | | | |
| | located in the middle of | | | | |
| | section of the vine. | | | | |
| | Generally sweet potatoes have | | | | |
| | 1, 3, 5, 7 or 9 leaf lobes. If the | | | | |
| | leaf has no lateral lobes but | | | | |
| | shows a central tooth this | | | | |
| | number is 1. If the apical | | | | |
| | portion of the leaf is totally | | | | |
| | rounded this number is 0. | | | | |
| Shape of | | 1 | Toothed | | |
| central leaf | | 2 | Triangular | | |
| lobe | | 3 | Semi-circular | | |
| | | 4 | Semi-elliptic | | |
| Mature leaf | Length from the basal lobes to | 5 | Medium | 8-15 | cm |
| size | the tip of the leaves. Record | | | | |
| | the average expression of at | | | | |
| | least 3 leaves located in the | | | | |
| | middle section of the vine. | | | | |
| Apxial leaf | Describe the most frequent | 5 | Main rib | | |
| vein | expression of the distribution | | partially purple | | |
| pigmentation | of anthocyanin (purple) | | | | |
| | pigmentation shown in the | | | | |
| | veins of the leaves lower | | | | |
| | surface | | | | |
| Mature leaf | Describe the overall foliage | 2 | Green | | |
| colour | colour considering the colour | | | | |
| | of the fully expanded mature | | | | |
| | leaves of several plants. The | | | | |
| | variegation in leaf colour due | | | | |
| | to virus symptoms should not | | | | |
| | be recorded. | | | | |
| Immature | Describe the overall foliage | 2 | Green | | |
| leaf colour | colour considering the colour | | | | |
| | of the fully expanded | | | | |
| | immature leaves of several | | | | |
| | plants. The variegation in leaf | | | | |
| | colour due to virus symptoms | | | | |
| | should not be recorded. | | | | |
| Petiole | Average petiol length, from | 5 | Intermediate | 21-30 | cm |
| length | the base to the insertion with | | | | |
| | the blade, of at least 3 leaves | | | | |
| | in the middle portion of the | | | | |
| | main vine. | | | | |
| Petiole | Distribution of the | 4 | Green with | | |
| pigmentation | anthocyanin (purple) | | purple at both | | |
| | pigmentation in the petioles of | | ends | | |
| | leaves. Indicate the most | | | | |
| | predominant colour first | | | | |
| Storage root | Storage root outline shown in | 2 | Round elliptic | slightly circular outline | |
| shape | longitudinal section. | 3 | Elliptic | with acute ends, and L/B | |
| | | 4 | Ovate | ratio not more than 2:1 | |
| | | 5 | Obvoate | symmetrical outline with | |
| | | 6 | Oblong | about the maximum | |
| | | 7 | Long Oblong | breadth at equal distance | |
| | | 8 | Long elliptic | from both ends which are | |
| | | | | slightly acute. L/B ratio | |
| | | | | not more than 3:1 | |
| | | | | outline resembling the | |
| | | | | longitudinal section of an | |
| | | | | egg. The broadest part is | |
| | | | | at the distal end (away | |
| | | | | from the root stalk) | |
| | | | | inversely ovate outline. | |
| | | | | The broadest part is at the | |
| | | | | proximal end (i.e. close to | |
| | | | | the root stalk) | |
| | | | | almost rectangular outline | |
| | | | | with sides nearly parallel | |
| | | | | and corners rounded. L/B | |
| | | | | ratio is 2:1. | |
| | | | | oblong outline with a L/B | |
| | | | | ratio about 2:1 | |
| Storage root | | 0 | Absent | | |
| surface | | | | | |
| defects | | | | | |
| Storage root | | 5 | Intermediate | 2-3 | mm |
| cortex | | | | | |
| thickness | | | | | |
| Predominant | | 9 | Dark-purple | | |
| skin colour | | | | | |
| Intensity of | | 3 | Dark | | |
| the | | | | | |
| predominant | | | | | |
| skin colour | | | | | |
| Secondary | | 8 | Purple-red | | |
| skin colour | | | | | |
| Predominant | | 9 | Strongly | | |
| flesh colour | | | pigmented with | | |
| | | | anthocyanine | | |
| Secondary | | 2 | Cream | | |
| flesh colour | | | | | |
| Distribution | | 6 | Ring and other | | |
| of secondary | | | areas in the | | |
| flesh colour | | | flesh | | |
| Flowering | | 5 | Moderate | | |
| habit | | | | | |
| Flower | | 4 | Pale purple | | |
| colour | | | limb with | | |
| | | | purple throat | | |
| Flower | | 5 to 6 | | | |
| length (cm) | | | | | |
| Flower | | 4 to 5 | | | |
| width (cm) | | | | | |
| Shape of | | 7 | Rounded | | |
| limb | | | | | |
| Equality of | | 1 | Outer two | | |
| sepal length | | | shorter | | |
| Number of | Number of veins observed in | 1 | | | |
| sepal veins | sepals. Record the most | | | | |
| | requent number in ten typical | | | | |
| | flowers | | | | |
| Sepal shape | | 1 | Ovate | | |
| | | 3 | Elliptic | | |
| | | 5 | Obovate | | |
| Sepal apex | | 1 | Acute | | |
| | | 3 | Obtuse | | |
| Sepal | | 0 | Absent | | |
| pubescence | | | | | |
| Sepal colour | | 5 | Green with | | |
| | | | purple areas | | |
| Colour of | | 5 | Pale purple | | |
| the stigma | | | | | |
| Colour of | | 3 | White with | | |
| style | | | purple at the | | |
| | | | base | | |
| Stigma | The relative position of the | 5 | Slightly exerted | | |
| exertion | stigma as compared to the | | | | |
| | highest anther. | | | | |
| Seed capsule | | 3 | Sparse | | |
| set | | | | | |
| Storage root | Arrangement of the storage | 1 | Closed cluster | | |
| formation | roots on the underground | 3 | Open cluster | | |
| | stems. | | | | |
| Storage root | Length of the stalk joining the | 5 | Intermediate | 6-8 | cm |

| | | | |
|---|---|---|---|
| stalk | storage roots to the stems | | |
| Number of | Average of ten plants | 10 | |
| storage roots | | | |
| per plant | | | |
| Variability | | 7 | Moderately |
| of storage | | | variable |
| root shape | | | |
| Variablilty | | 7 | Moderately |
| of storage | | | variable |
| root size | | | |
| Storage root | Average cracking shown in | 3 | Few cracks |
| cracking | ten plants. Consider all cracks | | |
| | caused by growth and/or water | | |
| | stress. Specify reference or | | |
| | reference cultivar | | |
| Latex | Amount of latex observed | 7 | Abundant |
| production | after cross sectioning | | |
| in storage | medium-sized storage roots | | |
| roots | | | |
| Oxidation in | Amount of browning due to | 5 | Some |
| storage roots | oxidation observed 5-10 | | |
| | seconds after storage roots are | | |
| | cut in cross. Sepicify in | | |
| | reference or reference cultivar | | |

‘P4’ has not been observed under all possible environmental conditions. Phenotype may vary due to environmental influence without variation in genotype. Sweetpotato line ‘P4’ shows uniformity and stability within the limits of environmental influence for the traits described herein. No variant traits have been observed or are expected in ‘P4’.

Color ratings were determined using the RHS Colour Chart of The Royal Horticultural Society of London (RHS), 2015 Edition, except where general color terms of ordinary significance are used. The following describe approximately field-established plants of 60 to 90 days after transplanting (DAT). Measurements and numerical values represent averages of typical plants. *These are typical values. Values may vary due to environment. Other values that are substantially equivalent are within the scope of the invention.
Botanical classification: Ipomoea batatas (L.) Lam
Common name—Sweetpotato
Variety name—‘P4’

### Parentage:

Female parent.—‘CH-Purple’, originally sprouted from sweetpotatoes sold in a local farmer's market, not patented.
Male parent.—‘Resisto’, characterized by its high yield and resistance to disease and insect pets, not patented.

### Plant Description:

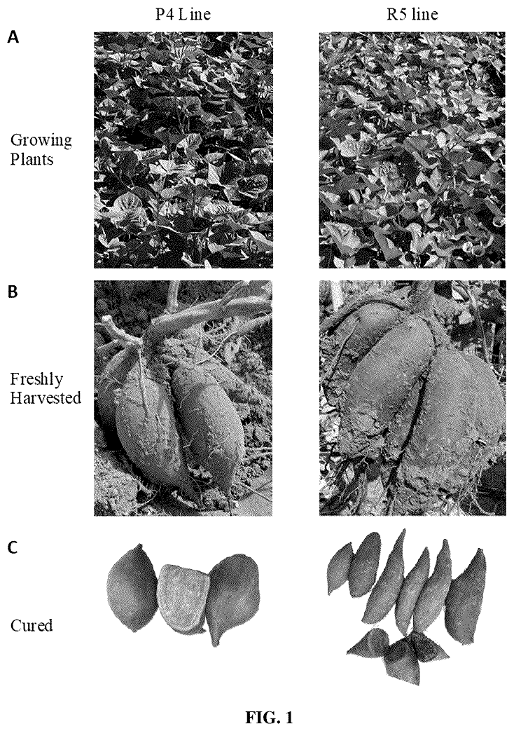

Growth conditions: ‘P4’ is a perennial herbaceous plant under frost-free warm climate conditions, but can be grown as an annual plant by vegetative propagation using either sprouts from storage roots (sweetpotatoes) or stem cuttings. ‘P4’ transplants grow rapidly in field under warm weather, and has a larger than 95% ground cover at 35-40 days after transplanting (DAT). ‘P4’ plants can tolerate extended periods (1 to 2 months) of high temperatures (35 to 40° C.), but will yield fewer storage roots per plant. ‘P4’ also grow very well in container culture.
Growth habit and general appearance: ‘P4’ has a predominantly prostrate growth habit with a vine system. The main vines are extremely spreading (>250 cm), and have many semi-erect young branches.
Above-ground structure and coloration: FIG. 1A shows the typical structured canopy, and shape and colorations of leaves of ‘P4’. The color of mature leaves of ‘P4’ is usually RHS 133B (deep green), although it may vary somewhat due to temperature and soil nutrient conditions. However, the first two to three young leaves of branches of ‘P4’ have a characteristic purple color even under extended periods (1 to 2 months) of high temperatures (35 to 40° C.). Leaves and branches of neighboring vines are layered to fill the gap, which together form a thick multiple-layered canopy for established plants. The extremely spreading vine canopy can reach a height of 60 to 80 cm, and a spread of large than 250 cm after growth for about 110 DAT.

### Branches:

Branching habit—Freely branching without the requirement of apex removal to stimulate branching, usually 3 to 5 main branches on the stem and 5 to 8 secondary branching on each main branch.
Stem—Appearance: Round and glabrous. Color: RHS 131B (Dark green) with RHS N79A (Purple) spots or strips and patches. Length: usually around 3 to 4 m; some can reach more than 5 m. Diameter: 7 to 9 mm. Internodes: short with an average length of about 3 to 5 cm; several lateral branches may be formed and each axil has latent shoots or flower buds Inflorescence).
New shoots—Appearance: Glabrous. Aspect: Undulating. Color: RHS 131B (Dark green) with heavy flushing near RHS N77A (purple) approaching the apex and at the base.
Vegetative secondary lateral branches—Length: About 1 to 3 m. Diameter: About 6-9 cm.
Internodes: short with an average length of about 6 to 9 cm; several lateral branches may be formed and each axil has latent shoots. Color: RHS 131B (Dark green) with RHS N79B (Purple) spots or strips and patches.
Petiole—Length: About 10 to 25 cm. Diameter: About 2 to 5 mm. Color: RHS 131B (Dark green) in the middle and RHS N79B (Purple) at both ends.

### Foliage Description:

Appearance and arrangement—very dense foliage with a matte finish; simple leaves are alternate and simple and tend to spiral around the stem.
Shape—Multi-shaped, including cordate heart-shaped, triangular, hastate tri-lobular and spear-shaped with the basal lobes more or less divergent, and lobed; mostly variable as is size.
Quantity—up to 25 or more leaves per lateral branch.
Mature leaf—Length: About 8 to 15 cm. Width: About 6 to 10 cm. Lobes: Central lobe shape: from toothed, triangular, semi-circular to semi-elliptic. Central lobe length: About 8 to 15 cm. Central lobe width: About 3 to 5 cm. Mid-vein lobe length: About 8 to 15 cm. Mid-vein lobe width: About 3 to 5 cm.
Apex—Predominantly acuminate, but can be acute, obtuse and mucronate.
Base—Hastate, sagittate and cordate, somewhat variable.
Margin—Entire.
Texture upper surface.—Glabrous.
Texture lower surface.—Glabrous.
Venation pattern.—Palmate at the base with arcuate veins in the center lamina.

### Flowering Description:

General—The photoperiod for flower induction is day neutral. Established plants in field flowers moderately for extended period under mild temperatures (e.g. early summer and fall). Pot-grown plants can flower under mild temperatures too. Flower sizes may vary depending on the growth conditions.
Type and arrangement—Two or four flowers in first or second order are arranged on a cyme inflorescence arising from leaf axils on main or secondary lateral branches.
Lastingness of flowers on the plant—Flowers may remain open for 2 to 4 days depending on temperature.
Fragrance—Slight.
Buds—Shape: lanceolate. Width: About 2 to 4 mm. Length: About 5 to 12 mm. Color: RHS N79B (purple).
Corolla—General: Composed of 5 fused petals which form a funnel with a rounded limb. Width: About 4 cm. Length: About 5 cm. Throat (tube): Outer throat: RHS 84C to 84D (Pinkish white). Inner throat: RHS 80A (purple). Petals: Fused to form a funnel shaped corolla with a rounded limb. Color, upper surface: RHS 84C (Pinkish white). Color, lower surface: RHS 84C (Pinkish white). Limb color: RHS 84C (Pinkish white) to RHS 84D (Pale pinkish white). Limb shape: Rounded.
Sepals—Quantity per flower: 5. General: The 5 sepals, 2 outer and 3 inner form the calyx. The two outer sepals are shorter than three the inner sepals. The calyx stay attached to the floral axle after the petals dry up and fall. Shape: The two shorter and long sepals are ovate and obovate, respectively. Apex: Acute to Obtuse. Length: About 5 mm. Width: About 2 mm. Color: Upper surface: RHS 140A (Green) with RHS 81A (Purple) spots. Lower surface: RHS 140C (Pale green).
Stigma—Color: RHS 84C (Pinkish white). Placement: Slightly exerted relative to the stamens. Length: About 5-8 mm. Width: About 2 mm.
Style color—RHS 155C (white) with RHS 77A (Purple) at the base.
Ovary—Superior with two locules that contain two or three ovules. At the base of the ovary there are basal glands containing nectar continuing halfway up the ovary. Ovary color: RHS 2D (yellow).
Stamen—Quantity per flower: 5. Anther length: About 2 to 3 mm. Anther color: RHS 84D (Pinkish white. Pollen production: moderate. Pollen color: Undetermined.

### Inflorescence Description:

General—The inflorescence is biparous cyme. The peduncle is divided in two axillary peduncles each of which is further divided in two. Four flower buds of different sizes in first and the second order of branching are more predominant.
Peduncle—Color: RHS N79A (purple). Length: About 1 to 6 cm. Diameter: About 2 to 3 mm.

### C. Deposit Information

A deposit of representative sample of plant tissue of sweetpotato line ‘P4’ was made with the the Provasoli-Guillard National Center for Marine Algae and Microbiota (NCMA), 60 Bigelow Drive, East Boothbay, Maine, 04544 USA. The deposit was assigned NCMA Accession No. ______. The date of deposit of the representative sample of plant tissue with the NCMA was ______. The deposit has been accepted under the Budapest Treaty and will be maintained in the NCMA depository for a period of 30 years, or 5 years after the most recent request, or for the enforceable life of the patent, whichever is longer, and will be replaced if necessary during that period. Upon issuance, all restrictions on the availability to the public of the deposit will be irrevocably removed consistent with all of the requirements of the Budapest Treaty and 37 C.F.R. §§ 1.801-1.809. Applicant does not waive any infringement of rights granted under this patent or under the Plant Variety Protection Act (7 USC 2321 et seq.).

### Sweetpotato Line ‘R5’

The disclosure provides Ipomoea batatas plants having purple-flesh, improved weevil-resistance, and self-compatibility,

### D. Origin and Breeding History

Sweetpotato line ‘R5’ was selected from among 264 germinated hybrids from a ‘Resisto’-mothered x ‘CH-Purple’ cross. During trials, plants of the selected line were surprisingly observed to yield purple-fleshed sweetpotatoes having commercially desirable slender-round shapes, a very good number of sweetpotatoes per plant (e.g. 8-12), and very good size uniformity (FIG. 1). Although sweetpotatoes produced by the ‘R5’ line have reduced anthocyanin content compared to those produced by the ‘P4’ line, the texture of the cooked ‘R5’ sweetpotatoes is improved, which is at least partly due to reduced dry-matter content as compared to those from the ‘CH-Purple’ line. These features have made the ‘R5’ line a very good candidate for producing purple-fleshed sweetpotatoes for fresh markets.

The most important feature of the ‘R5’ line is that it is a rare highly self-fertile line. It can produce copious numbers of seeds through natural pollination, which have enabled us to have generated a very large true F2 population including 600 germinated lines and more than 3000 seeds. Currently, this F2 population from the ‘R5’ line is one of its kind in sweetpotato. The ‘R5’ line also has a day-neutral photoperiod, and can produce copious flowers on well-grown vines under cool temperature (˜20 to 25° C.) all year round. Unlike most sweetpotato lines, the ‘R5’ line produces flowers having anthers higher than stigma, which could be one of the reasons for its self-compatibility. Additionally, the ‘R5’ line is of wider cross-compatibility. For example, ‘R5’ can be backcrossed to ‘Resisto’, and crossed to many F1 siblings and many other cultivars.

The male parent, ‘CH-Purple’, was originally sprouted from sweetpotatoes sold at the Waller County Farmers Market in Hempstead, TX. ‘CH-Purple’ is characterized by its purple flesh and reddish-purple skin. The female parent, ‘Resisto’ is characterized by its high yield and resistance to disease and insect pests.

Asexual reproduction of sweetpotato line ‘R5’ by sprouted plantlets or slips since May of 2020 has demonstrated that sweetpotato line ‘R5’ reproduces true-to-type for all of the physiological and morphological characteristics described herein. All of the physiological and morphological characteristics described herein are firmly fixed and retained through successive generations of such asexual propagation.

### E. Phenotypic Description

In accordance with another aspect of the present invention, there is provided an sweetpotato plant having the morphological characteristics of sweetpotato line ‘R5’. A description of the morphological and physiological characteristics of sweetpotato line ‘R5’ is presented below.

The following characteristics have been repeatedly observed and can be used to distinguish ‘R5’ as a new and distinct variety of Ipomoea batatas plant:

TABLE 3
Summary of Morphological and Physiological Characteristics of Sweetpotato line ‘R5’

| Characteristics | ‘R5’ |
|---|---|
| Leaves | Moderate-sized leaves with mostly 3-sharp lobs, light-green young and |
| | adult leaves. |
| Vines | Moderate length, mostly green-colored and moderate-diametered vines. |
| Photoperiod and | Day-neutral photoperiod, copious flowers under cool temperature. |
| Flowers | |
| Cross-compatibility | Self-fertile and producing many true seeds under cool temperatures, |
| | and cross-compatible to the parental lines and many siblings. |
| Roots (fibrous) | Reddish skin |
| Storage Roots | Reddish, smooth, and thin skin; purple flesh with spotty orange to |
| | whitish colors; 8 to 12 long round or oblong sweetpotatoes per plants |
| | and many jumbo-sized ones if grown over 110 days after planting. |
| Weevil Infection | 25 to 50% in non-treated but crop-rotated plots; and <1% in |
| | insecticide-treated plots. |
| Resistance Against | Much less susceptible to the southern potato wireworm (Conoderus |
| Other Soil Insects | falli Lane) and white grubs. |
| Leaf-eater | Very good. |
| Resistance | |
| Virus Resistance | Highly resistant against SPLCV |
| Other Notable | Resistant to fusarium wilt or stem rot |
| Disease Resistance | |

TABLE 4
International Board for Plant Genetic Resources (IBPGR) Sweetpotato Descriptors for Sweetpotato
line ‘R5’. (See Genesys (2018). Descriptors for sweet potato. Version 1991)

| Descriptor | | Coded | Title for | Additional | |
|---|---|---|---|---|---|
| Title | Description | Value | Coded Value | Description | |
| Twining | Ability of vines to climb | 0 | No-Twining | | |
| | adjacent stakes placed in those | | | | |
| | accessions showing twining | | | | |
| | characters | | | | |
| Plant type | Length of the main vines | 7 | Spreading | 151-250 | cm |
| Ground | Estimated percentage of the | 7 | High | 75-90% | |
| cover | ground cover recorded 35-40 | | | | |
| | days after planting | | | | |
| Vine | Average length of at least three | 3 | Short | 3-5 | cm |
| internode | internodes located in the | | | | |
| length | middle section of the vine. | | | | |
| Vine | Average diameter of at least | 3 | Thin | 4-6 | mm |
| internode | three internodes located in the | | | | |
| diameter | middle section of the vine. | | | | |
| Predominant | Anthocyanine (purple | 4 | Green with | | |
| vine colour | pigmentation) present in the | | many purple | | |
| | vines beside the green colour. | | spots | | |
| | The predominant colour should | | | | |
| | be evaluated considering the | | | | |
| | whole vine from base to tip. | | | | |
| Secondary | Anthocyanine (purple | 1 | Green base | | |
| vine colour | pigmentation) present in the | 2 | Green tip | | |
| | vines beside the green colour. | 4 | Purple base | | |
| | The predominant colour should | 6 | Purple nodes | | |
| | be evaluated considering the | | | | |
| | whole vine from base to tip. | | | | |
| | The secondary colour is more | | | | |
| | easily evaluated using younger | | | | |
| | vines. | | | | |
| Vine tip | Degree of hairiness of | 3 | Sparse | | |
| pubescence | immature leaves recorded at the | | | | |
| | apex of the vines | | | | |
| General | Described from mature leaves | 4 | Triangular | Heart-shaped | |
| outline of | located in the middle section of | 6 | Lobed | Trilobular and spear- | |
| the leaf | the vine. | | | shaped, with the basal | |
| | | | | lobes more or less | |
| Leaf lobes | | 1 | Very slight | Teeth | |
| type | | 3 | Slight | | |
| | | 5 | Moderate | | |
| Leaf lobe | Most leaves of sweet potato | 1 to 3 | | | |
| number | have two basal lobes and they | | | | |
| | should not be counted. Record | | | | |
| | the predominant number of the | | | | |
| | lateral and central leaf lobes | | | | |
| | observed on the leaves located | | | | |
| | in the middle of section of the | | | | |
| | vine. | | | | |
| | Generally sweet potatoes have | | | | |
| | 1, 3, 5, 7 or 9 leaf lobes. If the | | | | |
| | leaf has no lateral lobes but | | | | |
| | shows a central tooth this | | | | |
| | number is 1. If the apical | | | | |
| | portion of the leaf is totally | | | | |
| | rounded this number is 0. | | | | |
| Shape of | | 1 | Toothed | | |
| central leaf | | 2 | Triangular | | |
| lobe | | 3 | Semi-circular | | |
| | | 4 | Semi-elliptic | | |
| Mature leaf | Length from the basal lobes to | 5 | Medium | 8-15 | cm |
| size | the tip of the leaves. Record the | | | | |
| | average expression of at least 3 | | | | |
| | leaves located in the middle | | | | |
| | section of the vine. | | | | |
| Apxial leaf | Describe the most frequent | 8 | All veins | | |
| vein | expression of the distribution of | | mostly or | | |
| pigmentation | anthocyanin (purple) | | totally purple | | |
| | pigmentation shown in the | | | | |
| | veins of the leaves lower | | | | |
| | surface | | | | |
| Mature leaf | Describe the overall foliage | 3 | Green with | | |
| colour | colour considering the colour | | purple edges | | |
| | of the fully expanded mature | | | | |
| | leaves of several plants. The | | | | |
| | variegation in leaf colour due | | | | |
| | to virus symptoms should not | | | | |
| | be recorded. | | | | |
| Immature | Describe the overall foliage | 1 | Yellow-green | | |
| leaf colour | colour considering the colour | 3 | Green wiith | | |
| | of the fully expanded immature | | purple edges | | |
| | leaves of several plants. The | | | | |
| | variegation in leaf colour due | | | | |
| | to virus symptoms should not | | | | |
| | be recorded. | | | | |
| Petiole | Average petiol length, from the | 3 | Short | 10-20 | |
| length | base to the insertion with the | | | | |
| | blade, of at least 3 leaves in the | | | | |
| | middle portion of the main | | | | |
| | vine. | | | | |
| Petiole | Distribution of the anthocyanin | 2 | Green with | | |
| pigmentation | (purple) pigmentation in the | | purple near | | |
| | petioles of leaves. Indicate the | | stem | | |
| | most predominant colour first | 3 | Green with | | |
| | | | purple near | | |
| | | | leaf | | |
| | | 4 | Green with | | |
| | | | purple at both | | |
| | | | ends | | |
| Storage root | Storage root outline shown in | 2 | Round elliptic | slightly circular outline | |
| shape | longitudinal section. | 3 | Elliptic | with acute ends, and L/B | |
| | | 8 | Long elliptic | ratio not more than 2:1 | |
| | | | | symmetrical outline with | |
| | | | | about the maximum | |
| | | | | breadth at equal distance | |
| | | | | from both ends which are | |
| | | | | slightly acute. L/B ratio | |
| | | | | not more than 3:1 | |
| | | | | oblong outline with a L/B | |
| | | | | ratio about 2:1 | |
| Storage root | | 0 | Absent | | |
| surface | | | | | |
| defects | | | | | |
| Storage root | | 7 | Thick | 3-4 | mm |
| cortex | | | | | |
| thickness | | | | | |
| Predominant | | 8 | Purple-red | | |
| skin colour | | | | | |
| Intensity of | | 2 | Intermediate | | |
| the | | | | | |
| predominant | | | | | |
| skin colour | | | | | |
| Secondary | | 9 | Dark-purple | | |
| skin colour | | | | | |
| Predominant | | 9 | Strongly | | |
| flesh colour | | | pigmented | | |
| | | | with | | |
| | | | anthocyanine | | |
| Secondary | | 2 | Cream | | |
| flesh colour | | 3 | Yellow | | |
| | | 4 | Orange | | |
| Distribution | | 3 | Scattered spots | | |
| of secondary | | | in flesh | | |
| flesh colour | | | | | |
| Flowering | | 7 | Profuse | | |
| habit | | | | | |
| Flower | | 2 | White limb | | |
| colour | | | with purple | | |
| | | | throat | | |
| Flower | | 3 to 5 | | | |
| length (cm) | | | | | |
| Flower | | 3 to 4 | | | |
| width (cm) | | | | | |
| Shape of | | 7 | Rounded | | |
| limb | | | | | |
| Equality of | | 1 | Outer two | | |
| sepal length | | | shorter | | |
| Number of | Number of veins observed in | 1 | | | |
| sepal veins | sepals. Record the most requent | | | | |
| | number in ten typical flowers | | | | |
| Sepal shape | | 3 | Elliptic | | |
| | | 5 | Obovate | | |
| Sepal apex | | 3 | Obtuse | | |
| Sepal | | 3 | Sparse | | |
| pubescence | | | | | |
| Sepal colour | | 1 | Green | | |
| Colour of | | 1 | White | | |
| the stigma | | | | | |
| Colour of | | 3 | White with | | |
| style | | | purple at the | | |
| | | | base | | |
| Stigma | The relative position of the | 1 | Inserted | | |
| exertion | stigma as compared to the | | | | |
| | highest anther. | | | | |
| Seed capsule | | 7 | Profuse | | |
| set | | | | | |
| Storage root | Arrangement of the storage | 1 | Closed cluster | | |
| formation | roots on the underground | 3 | Open cluster | | |
| | stems. | | | | |
| Storage root | Length of the stalk joining the | 5 | Intermediate | 6-8 | cm |

| | | | |
|---|---|---|---|
| stalk | storage roots to the stems | | |
| Number of | Average of ten plants | 12 | |
| storage roots | | | |
| per plant | | | |
| Variability | | 5 | Slightly |
| of storage | | | variable |
| root shape | | | |
| Variablilty | | 7 | Moderately |
| of storage | | | variable |
| root size | | | |
| Storage root | Average cracking shown in ten | 0 | Absent |
| cracking | plants. Consider all cracks | | |
| | caused by growth and/or water | | |
| | stress. Specify reference or | | |
| | reference cultivar | | |
| Latex | Amount of latex observed after | 3 | Little |
| production | cross sectioning medium-sized | | |
| in storage | storage roots | | |
| roots | | | |
| Oxidation in | Amount of browning due to | 3 | Little |
| storage roots | oxidation observed 5-10 | | |
| | seconds after storage roots are | | |
| | cut in cross. Sepicify in | | |
| | reference or reference cultivar | | |

‘R5’ has not been observed under all possible environmental conditions. Phenotype may vary due to environmental influence without variation in genotype. Sweetpotato line ‘R5’ shows uniformity and stability within the limits of environmental influence for the traits described herein. No variant traits have been observed or are expected in ‘R5’.

Color ratings were determined using the RHS Colour Chart of The Royal Horticultural Society of London (RHS), 2015 Edition, except where general color terms of ordinary significance are used. The following describe approximately field-established plants of 60 to 90 days after transplanting (DAT). Measurements and numerical values represent averages of typical plants. *These are typical values. Values may vary due to environment. Other values that are substantially equivalent are within the scope of the invention.
Botanical classification: Ipomoea batatas (L.) Lam
Common name—Sweetpotato
Variety name—‘R5’
Parentage:
Female parent.—‘Resisto’, characterized by its high yield and resistance to disease and insect pets, not patented.
Male parent.—‘CH-Purple’, a farm variety, originally sprouted from sweetpotatoes sold in a local farmer's market, not patented.

### Plant Description:

Growth conditions: ‘R5’ is a perennial herbaceous plant under frost-free warm climate conditions, but can be grown as an annual plant by vegetative propagation using either sprouts from storage roots (sweetpotatoes) or stem cuttings. ‘R5’ transplants grow rapidly in field under warm weather, and has a 75-90% ground cover rate at 35-40 days after transplanting (DAT). ‘R5’ plants can tolerate extended periods (1 to 2 months) of high temperatures (35 to 40° C.) without reduction in the number of storage roots per plant, but will yield storage roots of much smaller sizes. ‘P4’ also grow very well in container culture.
Growth habit and general appearance: ‘R5’ has a predominantly prostrate growth habit with a vine system. The main vines are spreading (150 to 250 cm), and have many semi-erect young branches.
Above-ground structure and coloration: FIG. 1, Panel B shows the typical structured canopy, and shape and colorations of leaves of ‘R5’. The color of mature leaves of ‘R5’ is usually RHS 134B (bright green,) although it may vary somewhat due to temperature and soil nutrient conditions. Leaves and branches of neighboring vines are layered to fill the gap, which together form a medium thick multiple-layered canopy for established plants. The spreading vine canopy can reach a height of 40 to 60 cm, and a spread of 150 to 250 cm after growth for about 110 DAT.

### Branches:

Branching habit—Freely branching without the requirement of apex removal to stimulate branching, usually 5 to 6 main branches on the stem and 3 to 5 secondary branching on each main branch.
Stem—Appearance: Sparsely to moderately pubescent. Color: RHS 132A (Dark green) on the upper parts and RHS N79A (Purple) on the shaded lower parts and at the base. Length: usually between 150 to 250 cm. Diameter: 4 to 6 mm. Internodes: short with an average length of about 3 to 5 cm; several lateral branches may be formed and each axil has latent shoots or flower buds (Inflorescence).
New shoots—Appearance: Sparsely to moderately pubescent. Aspect: Undulating. Color: RHS 132A (Dark green) on the upper part and RHS N79A (Purple) on the shaded lower parts and at the base.
Vegetative secondary lateral branches—Length: About 150 to 250 cm. Diameter: About 4-6 mm. Internodes: short with an average length of about 3 to 5 cm; several lateral branches may be formed, and each axil has latent shoots and/or flower buds (Inflorescence). Color: RHS 132A (Dark green) on the upper parts and RHS N79A (Purple) on the shaded lower parts and at the base.
Petiole—Length: About 10 to 20 cm. Diameter: About 2 to 5 mm. Color: RHS 132A (Dark green) on the upper parts and RHS N79A (Purple) on shaded parts and at both ends.

### Foliage Description:

Appearance and arrangement—very dense foliage with a semi-glossy finish; simple leaves are alternate and simple and tend to spiral around the stem.
Shape—Multi-shaped, including triangular, hastate tri-lobular and spear-shaped with the basal lobes more or less divergent, and lobed; mostly variable as is size.
Quantity—up to 30 or more leaves per lateral branch.
Mature leaf—Length: About 4 to 10 cm. Width: About 3 to 7 cm. Lobes: Central lobe shape: from toothed, triangular, semi-circular to semi-elliptic. Central lobe length: About 4 to 10 cm. Central lobe width: About 1 to 3 cm. Mid-vein lobe length: About 4 to 10 cm. Mid-vein lobe width: About 2 to 4 cm.
Apex—Predominantly acuminate, but can be acute, obtuse and mucronate.
Base—Predominantly obtuse and cordate, somewhat variable.
Margin—Entire.
Texture upper surface.—Glabrous.
Texture lower surface.—Glabrous.
Venation pattern.—Palmate at the base with arcuate veins in the center lamina.

### Flowering Description:

General—The photoperiod for flower induction is day neutral. Established plants in field flowers profusely for extended period under mild temperatures in all seasons. Pot-grown plants can flower profusely under mild temperatures too. Flower sizes may vary depending on the growth conditions.
Type and arrangement—Two, four and even six flowers in first, second and third order are arranged on a cyme inflorescence arising from leaf axils on main or secondary lateral branches.
Lastingness of flowers on the plant—Flowers may remain open for 2 to 4 days depending on temperature.
Fragrance—Slight.
Buds—Shape: lanceolate. Width: About 2 to 4 mm. Length: About 5 to 12 mm. Color: RHS 140C (Pale green).
Corolla—General: Composed of 5 fused petals which form a funnel with a rounded limb. Width: About 3 cm. Length: About 4 cm. Throat (tube): Outer throat: RHS 84C to 84D (Pinkish white). Inner throat: RHS 80A (purple). Petals: Fused to form a funnel shaped corolla with a rounded limb. Color, upper surface: RHS 84C (Pinkish white). Color, lower surface: RHS 84C (Pinkish white). Limb color: RHS 84C (Pinkish white) to RHS 84D (Pale pinkish white). Limb shape: Rounded.
Sepals—Quantity per flower: 5. General: The 5 sepals, 2 outer and 3 inner form the calyx. The two outer sepals are shorter than three the inner sepals. The calyx stay attached to the floral axle after the petals dry up and fall. Shape: The two shorter and long sepals are elliptic and obovate, respectively. Apex: Obtuse. Length: About 5 mm. Width: About 2 mm. Color: Upper surface: RHS 140A (Green). Lower surface: RHS 140C (Pale green).
Stigma—Color: RHS 84D (white). Placement: Inserted relative to the stamens. Length: About 5-8 mm. Width: About 2 mm.
Style color—RHS 155C (white) with RHS 81A (Purple) at the base.
Ovary—Superior with two locules that contain two or three ovules. At the base of the ovary there are basal glands containing nectar continuing halfway up the ovary. Ovary color: RHS 2D (yellow).
Stamen—Quantity per flower: 5. Anther length: About 2 to 3 mm. Anther color: RHS 84D (Pinkish white). Pollen production: profuse. Pollen color: Undetermined.

### Inflorescence Description:

General—The inflorescence is biparous cyme. The peduncle is divided in two axillary peduncles each of which is further divided in two. Four flower buds of different sizes in first and the second order of branching are more predominant.
Peduncle—Color: RHS 134A (Green). Length: About 2 to 8 cm. Diameter: About 2 to 3 mm.

### F. Deposit Information

A deposit of representative sample of plant tissue of sweetpotato line ‘R5’ was made with the the Provasoli-Guillard National Center for Marine Algae and Microbiota (NCMA), 60 Bigelow Drive, East Boothbay, Maine, 04544 USA. The deposit was assigned NCMA Accession No. ______. The date of deposit of the representative sample of plant tissue with the NCMA was ______. The deposit has been accepted under the Budapest Treaty and will be maintained in the NCMA depository for a period of 30 years, or 5 years after the most recent request, or for the enforceable life of the patent, whichever is longer, and will be replaced if necessary during that period. Upon issuance, all restrictions on the availability to the public of the deposit will be irrevocably removed consistent with all of the requirements of the Budapest Treaty and 37 C.F.R. §§ 1.801-1.809. Applicant does not waive any infringement of rights granted under this patent or under the Plant Variety Protection Act (7 USC 2321 et seq.).

### FURTHER EMBODIMENTS OF THE INVENTION

### A. Plant Breeding

In one aspect, the present disclosure provides plants modified using the methods described herein to include at least a first desired heritable trait. Such plants may, in one embodiment, be developed by backcrossing, wherein essentially all of the desired morphological and physiological characteristics of a cultivar are recovered in addition to a genetic locus transferred into the plant via the backcrossing technique. The term single locus converted plant as used herein refers to those plants which are developed by backcrossing or by genetic engineering, wherein essentially all of the desired morphological and physiological characteristics of a cultivar are recovered in addition to the single locus transferred into the cultivar via the backcrossing or genetic engineering technique, respectively. By essentially all of the desired morphological and physiological characteristics, it is meant that the characteristics of a plant are recovered that are otherwise present when compared in the same environment, other than an occasional variant trait that might arise during backcrossing, direct introduction of a transgene, or application of genetic engineering technique.

Backcrossing methods can be used with the present invention to improve or introduce a trait into a cultivar (also known as a line and a variety). The term backcrossing as used herein refers to the repeated crossing of a hybrid progeny back to one of the parental Ipomoea batatas plants. The parental Ipomoea batatas plant that contributes the locus or loci for the desired trait is termed the nonrecurrent or donor parent. This terminology refers to the fact that the nonrecurrent parent is used one time in the backcross protocol and therefore does not recur. The recurrent parent therefore provides the desired genetic background, while the choice of the particular nonrecurrent parent will depend on the purpose of the backcross. One of the major purposes is to add some commercially desirable trait to the plant. The exact backcrossing protocol will depend on the characteristic or trait being altered and the genetic distance between the recurrent and nonrecurrent parents. Although backcrossing methods are simplified when the characteristic being transferred is a dominant allele, a recessive allele or an additive allele (between recessive and dominant) may also be transferred. In this instance it may be necessary to introduce a test of the progeny to determine if the desired characteristic has been successfully transferred. The backcross process may be accelerated by the use of genetic markers, such as SSR, RFLP, SNP or AFLP markers to identify plants with the greatest genetic complement from the recurrent parent.

Modified backcrossing may also be used with sweetpotato plants comprising a weevil resistance trait, a purple flesh trait, and/or a self-fertility trait, such as those described herein. This technique uses different recurrent parents during the backcrossing. Modified backcrossing may be used to replace the original recurrent parent with a variety having certain more desirable characteristics or multiple parents may be used to obtain different desirable characteristics from each.

With the development of molecular markers associated with particular traits, it is possible to add additional traits into an established germ line, such as represented here, with the end result being substantially the same base germplasm with the addition of a new trait or traits. Molecular breeding, as described in Moose and Mumm, 2008 (Plant Physiol., 147: 969-977), for example, and elsewhere, provides a mechanism for integrating single or multiple traits or QTL into an elite line. This molecular breeding-facilitated movement of a trait or traits into an elite line may encompass incorporation of a particular genomic fragment associated with a particular trait of interest into the elite line by the mechanism of identification of the integrated genomic fragment with the use of flanking or associated marker assays. In the embodiment represented here, one, two, three or four genomic loci, for example, may be integrated into an elite line via this methodology. When this elite line containing the additional loci is further crossed with another parental elite line to produce hybrid offspring, it is possible to then incorporate at least eight separate additional loci into the hybrid. In one embodiment, each locus may confer a separate trait. In another embodiment, loci may need to be homozygous and exist in each parent line to confer a trait in the hybrid. In yet another embodiment, multiple loci may be combined to confer a single robust phenotype of a desired trait.

Many traits have been identified that are not regularly selected for in the development of a new variety but that can be improved by backcrossing techniques. A genetic locus conferring the traits may or may not be transgenic. Examples of such traits known to those of skill in the art include, but are not limited to, herbicide tolerance, disease resistance, pest resistance, and purple flesh color. These genes are generally inherited through the nucleus, but may be inherited through the cytoplasm.

Selection of Ipomoea batatas plants for breeding is not necessarily dependent on the phenotype of a plant and instead can be based on genetic investigations. For example, one can utilize a suitable genetic marker which is closely genetically linked to a trait of interest. One of these markers can be used to identify the presence or absence of a trait in the offspring of a particular cross and can be used in selection of progeny for continued breeding. This technique is commonly referred to as marker assisted selection. Any other type of genetic marker or other assay which is able to identify the relative presence or absence of a trait of interest in a plant can also be useful for breeding purposes. Procedures for marker assisted selection are well known in the art. Such methods will be of particular utility in the case of recessive traits and variable phenotypes, or where conventional assays may be more expensive, time consuming, or otherwise disadvantageous. In addition, marker assisted selection may be used to identify plants comprising desirable genotypes at the seed, seedling, or plant stage, to identify or assess the purity of a cultivar, to catalog the genetic diversity of a germplasm collection, and to monitor specific alleles or haplotypes within an established cultivar.

Types of genetic markers which could be used in accordance with the invention include, but are not necessarily limited to, Simple Sequence Length Polymorphisms (SSLPs) (Williams et al., Nucleic Acids Res., 1 8:6531-6535, 1990), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs) (EP 534 858, specifically incorporated herein by reference in its entirety), and Single Nucleotide Polymorphisms (SNPs) (Wang et al., Science, 280:1077-1082, 1998).

In particular embodiments of the invention, marker assisted selection is used to increase the efficiency of a backcrossing breeding scheme for producing an Ipomoea batatas line comprising a desired trait. This technique is commonly referred to as marker assisted backcrossing (MABC). This technique is well-known in the art and may involve, for example, the use of three or more levels of selection, including foreground selection to identity the presence of a desired locus, which may complement or replace phenotype screening protocols; recombinant selection to minimize linkage drag; and background selection to maximize recurrent parent genome recovery.

### B. Genetically Identifying Sweetpotato Lines

In an embodiment, the present invention provides a sweetpotato line characterized by the molecular and physiological data obtained from a representative sample of said line deposited with the Provasoli-Guillard National Center for Marine Algae and Microbiota (NCMA). Thus, plants, seeds, or parts thereof, having all of the morphological and physiological characteristics of sweetpotato line ‘P4’ or ‘R5’ are provided.

In some examples, a plant, a plant part, or a seed of sweetpotato line ‘P4’ or ‘R5’ may be characterized by producing a molecular profile. A molecular profile may include, but is not limited to, one or more genotypic and/or phenotypic profile(s). A genotypic profile may include, but is not limited to, a marker profile, such as a genetic map, a linkage map, a trait maker profile, a SNP profile, an SSR profile, a genome-wide marker profile, a haplotype, or the like. A molecular profile may also be a nucleic acid sequence profile, and/or a physical map. A phenotypic profile may include, but is not limited to, a protein expression profile, a metabolic profile, an mRNA expression profile, and the like.

One means of generating genetic marker profiles is to assay SNPs that are known in the art. Tens of thousands of SNPs are known in sweetpotato, see, e.g., Genetic Diversity and Population Structure of the USDA Sweetpotato (Ipomoea batatas) Germplasm Collections Using GBSpoly. Front. Plant Sci., 9 Aug. 2018. doi: 10.3389/fpls.2018.01166. A marker system based on SNPs can be highly informative in linkage analysis relative to other marker systems, in that multiple alleles may be present. Another advantage is that SNPs can be detected through use of strategically designed primers, probes, or other specially designed hybridization molecules, which eliminates the need to perform labor-intensive Southern blots. Further, many SNP detection methods are easily scalable and therefore can easily integrate into high-throughput analysis platforms such as microarray and next-generation sequencing technologies. High density microarray platforms, for example, are capable of analyzing hundreds of thousands SNPs on a single microarray chip.

A genotypic profile of sweetpotato line ‘P4’ or ‘R5’ can be used to identify a plant or population of plants comprising line ‘P4’ or ‘R5’ as a parent, because such plants will comprise the same allelic profile as line ‘P4’ or ‘R5’ at an expected frequency by Mendelian inheritance. In addition, plants and plant parts substantially benefiting from the use of sweetpotato line ‘P4’ or ‘R5’ in their development, such as sweetpotato line ‘P4’ or ‘R5’ comprising a backcross conversion, transgene, and the like, may be identified by having a molecular marker profile with a high percent identity to sweetpotato line ‘P4’ or ‘R5’.

A genotypic profile of sweetpotato line ‘P4’ or ‘R5’ also can be used to identify essentially derived varieties and other progeny varieties developed from the use of sweetpotato line ‘P4’ or ‘R5’, as well as cells and other plant parts thereof. Plants of the invention include any plant having at least 90%, 91%, 92%, 93%, 94%, 95%, 96%, 97%, 98%, 99%, 99.5%, or 99.9% of the markers in the genotypic profile, and that retain 90%, 91%, 92%, 93%, 94%, 95%, 96%, 97%, 98%, 99%, 99.5%, or 99.9% of the morphological and physiological characteristics of sweetpotato line ‘P4’ or ‘R5’ when grown under the same conditions. Such plants maybe developed using markers well known in the art. Progeny plants and plant parts produced using sweetpotato line ‘P4’ or ‘R5’ may be identified by any means known in the art that is indicative or consistent with the line. For example, progeny plants and plant parts produced using sweetpotato line ‘P4’ or ‘R5’ may be identified by having a molecular marker profile of at least 25%, 30%, 35%, 40%, 45%, 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% genetic contribution from sweetpotato line ‘P4’ or ‘R5’ by percent identity or percent similarity, or such plants may be identified by statistical genetic parameters. Unique molecular profiles may be identified with other next-generation sequencing tools, such as SNP discovery or haplotype analysis.

### C. Breeding of Sweetpotato Lines ‘P4’ and ‘R5’

The development of new varieties using one or more starting varieties is well known in the art and encompassed by the disclosure. In accordance with the disclosure, novel varieties may be created by crossing a plant of the disclosure followed by multiple generations of breeding according to such well-known methods. New varieties may be created by crossing with any second, compatible plant. New varieties may be developed, for example, by applying a breeding technique to a plant of sweetpotato line ‘P4’ or ‘R5’. Such breeding techniques are well-known in the art and include but are not limited to recurrent selection, mass selection, hybridization, open-pollination, backcrossing, modified backcrossing, pedigree breeding, mutation breeding, and marker assisted selection. “Mutation breeding” as used herein refers to a breeding technique comprising selecting a naturally occurring (spontaneous) mutation or inducing a mutation through means such as irradiation or chemical induction.

In selecting a second plant to cross with a plant of the disclosure, it will typically be preferred to choose those plants which either themselves exhibit one or more selected desirable characteristics or which exhibit the desired characteristic(s) when in hybrid combination. Once initial crosses have been made, selection takes place to produce new varieties. Examples of desirable traits may include, in specific embodiments, self- or cross-compatibility, flesh color, sweetpotato size, color patterning, foliage quality, shape and uniformity, maturity date, sweetpotato yield, seed germination rate, seedling vigor, pest and disease resistance, herbicide tolerance, and adaptability for soil and climate conditions. Consumer-driven traits are other traits that may be incorporated into new plants developed by this disclosure.

One aspect of the current disclosure therefore provides methods for producing an Ipomoea batatas plant comprising a purple flesh trait, a self-fertility trait, and/or improved weevil resistance. In certain embodiments, the method may comprise (a) producing a sweetpotato line ‘P4’-derived sweetpotato plant, or a sweetpotato line ‘R5’-derived sweetpotato plant from a seed produced by crossing a plant of sweetpotato line ‘P4’ or ‘R5’ with itself or a second sweetpotato plant; (b) crossing the sweetpotato line ‘P4’-derived or ‘R5’-derived sweetpotato plant with itself or a different sweetpotato plant to obtain a seed of a further sweetpotato line ‘P4’-derived or ‘R5’-derived sweetpotato plant; (c) selecting a further sweetpotato line ‘P4’-derived or ‘R5’-derived sweetpotato plant that comprises the purple flesh trait, the self-fertility trait, and/or improved weevil resistance; (d) repeating said producing, crossing, and selecting steps of (a), (b), and (c) using the seed of said step (b) for at least one generation to produce a seed of an additional line ‘P4’-derived or ‘R5’-derived sweetpotato plant; and (e) selecting an additional line ‘P4’-derived or ‘R5’-derived sweetpotato plant comprising the purple flesh trait, the self-fertility trait, and/or improved weevil resistance. In a particular embodiment, the second plant may be an Ipomoea batatas plant and the progeny seed may be planted and grown to produce fertile hybrid progeny plants. A plant in accordance with the disclosure may be used in such crosses as the female plant or the male plant.

The disclosure also provides methods of producing sweetpotato plants derived from sweetpotato line ‘P4’ or ‘R5’. The method may comprise (a) crossing a sweetpotato plant of sweetpotato line ‘P4’ or ‘R5’ with itself or a second plant capable of being crossed thereto; and (b) collecting resulting seed. In one embodiment, the second plant may be an Ipomoea batatas plant. In some embodiments, the methods of the present disclosure may further comprise the step of (c) crossing a plant grown from said seed of step (b) with itself or a second plant at least one or more additional time(s) to yield additional seed. Plants, seeds, and plant parts produced from the methods described herein and plants comprising the purple flesh trait, the self-fertility trait (i.e. self-compatibility), and/or improved weevil resistance as described herein are also provided.

In certain embodiments, hybrid seeds may be produced using the methods of the present disclosure. A parent plant of such a seed may be an Ipomoea batatas plant of sweetpotato line ‘P4’ or ‘R5’. In other embodiments, a plant as described herein may be either the male plant or the female plant in a given cross.

In accordance with the disclosure, many species of Ipomoea may be used. In particular, Ipomoea species that may be useful include but are not limited to I. lacunosa, I. triloba, I. trichocarpa, I. tiliacea and I. gracilis, I. batatas, I. trifida and I. litoralis, and the like.

### D. Plants Derived by Genetic Engineering

Various genetic engineering technologies have been developed and may be used by those of skill in the art to introduce traits in plants. In certain aspects of the claimed invention, traits are introduced into sweetpotato plants via altering or introducing a single genetic locus or transgene into the genome of a recited variety or progenitor thereof. Methods of genetic engineering to modify, delete, or insert genes and polynucleotides into the genomic DNA of plants are well-known in the art.

In specific embodiments of the invention, improved sweetpotato lines can be created through the site-specific modification of a plant genome. Methods of genetic engineering include, for example, utilizing sequence-specific nucleases such as zinc-finger nucleases (see, for example, U.S. Pat. Appl. Pub. No. 2011-0203012); engineered or native meganucleases; TALE-endonucleases (see, for example, U.S. Pat. Nos. 8,586,363 and 9,181,535); and RNA-guided endonucleases, such as those of the CRISPR/Cas systems (see, for example, U.S. Pat. Nos. 8,697,359 and 8,771,945 and U.S. Pat. Appl. Pub. No. 2014-0068797). One embodiment of the invention thus relates to utilizing a nuclease or any associated protein to carry out genome modification. This nuclease could be provided heterologously within donor template DNA for templated-genomic editing or in a separate molecule or vector. A recombinant DNA construct may also comprise a sequence encoding one or more guide RNAs to direct the nuclease to the site within the plant genome to be modified. Further methods for altering or introducing a single genetic locus include, for example, utilizing single-stranded oligonucleotides to introduce base pair modifications in an sweetpotato plant genome (see, for example Sauer et al., Plant Physiol, 170(4):1917-1928, 2016 and Wang et al., Int J Mol Sci. 20(19): 4702, 2019).

Methods for site-directed alteration or introduction of a single genetic locus are well-known in the art and include those that utilize sequence-specific nucleases, such as the aforementioned, or complexes of proteins and guide-RNA that cut genomic DNA to produce a double-strand break (DSB) or nick at a genetic locus. As is well-understood in the art, during the process of repairing the DSB or nick introduced by the nuclease enzyme, a donor template, transgene, or expression cassette polynucleotide may become integrated into the genome at the site of the DSB or nick. The presence of homology arms in the DNA to be integrated may promote the adoption and targeting of the insertion sequence into the plant genome during the repair process through homologous recombination or non-homologous end joining (NHEJ).

In another embodiment of the invention, genetic transformation may be used to insert a selected transgene into a plant of the disclosure or may, alternatively, be used for the preparation of transgenes which can be introduced by backcrossing. Methods for the transformation of plants that are well known to those of skill in the art and applicable to many plant species include, but are not limited to, electroporation, microprojectile bombardment, Agrobacterium-mediated transformation and direct DNA uptake by protoplasts. For example, Newell, et al., describes Agrobacterium-mediated transformation of sweetpotato (Plant Science, 107(2); 215-227. 1995).

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner.

An efficient method for delivering transforming DNA segments to plant cells is microprojectile bombardment. In this method, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target cells. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large. Microprojectile bombardment techniques are widely applicable, and may be used to transform virtually any plant species.

Agrobacterium-mediated transfer is another widely applicable system for introducing gene loci into plant cells. An advantage of the technique is that DNA can be introduced into whole plant tissues, thereby bypassing the need for regeneration of an intact plant from a protoplast. Modern Agrobacterium transformation vectors are capable of replication in E. coli as well as Agrobacterium, allowing for convenient manipulations. Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes.

Additionally, Agrobacterium containing both armed and disarmed Ti genes can be used for transformation.

In those plant strains where Agrobacterium-mediated transformation is efficient, it is the method of choice because of the facile and defined nature of the gene locus transfer. The use of Agrobacterium-mediated plant integrating vectors to introduce DNA into plant cells is well known in the art (U.S. Pat. No. 5,563,055, incorporated herein by reference in its entirety).

Transformation of plant protoplasts also can be achieved using methods based on calcium phosphate precipitation, polyethylene glycol treatment, electroporation, and combinations of these treatments.

A number of promoters have utility for plant gene expression for any gene of interest including but not limited to selectable markers, scoreable markers, genes for pest and disease resistance, and any other gene of agronomic interest. Examples of constitutive promoters useful for driving gene expression in plants include, but are not limited to, the cauliflower mosaic virus (CaMV) P-35S promoter, which confers constitutive, high-level expression in most plant tissues, including monocots; a tandemly duplicated version of the CaMV 35S promoter, the enhanced 35S promoter (P-e35S) the nopaline synthase promoter, the octopine synthase promoter; and the figwort mosaic virus (P-FMV) promoter as described in U.S. Pat. No. 5,378,619 (incorporated herein by reference in its entirety), and an enhanced version of the FMV promoter (P-eFMV) where the promoter sequence of P-FMV is duplicated in tandem, the cauliflower mosaic virus 19S promoter, a sugarcane bacilliform virus promoter, a commelina yellow mottle virus promoter, and other plant DNA virus promoters known to express in plant cells.

A variety of plant gene promoters that are regulated in response to environmental, hormonal, chemical, and/or developmental signals can be used for expression of an operably linked gene in plant cells, including promoters regulated by (1) heat, (2) light (e.g., pea rbcS-3A promoter; maize rbcS promoter; or chlorophyll a/b-binding protein promoter), (3) hormones, such as abscisic acid, (4) wounding (e.g., wunl); or (5) chemicals such as methyl jasmonate, salicylic acid, or Safener. It may also be advantageous to employ organ-specific promoters.

Exemplary nucleic acids which may be introduced to the plants of this disclosure include, for example, DNA sequences or genes from another species, or even genes or sequences which originate with or are present in the same species, but are incorporated into recipient cells by genetic engineering methods rather than classical reproduction or breeding techniques. However, the term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the plant cell, DNA from another plant, DNA from a different organism, or a DNA generated externally, such as a DNA sequence containing an antisense message of a gene, or a DNA sequence encoding a synthetic or modified version of a gene.

Many hundreds if not thousands of different genes are known and could potentially be introduced into a plant of sweetpotato line ‘P4’ or ‘R5’. Non-limiting examples of particular genes and corresponding phenotypes one may choose to introduce into a plant of the disclosure include one or more genes for insect tolerance, such as a Bacillus thuringiensis (B.t.) gene, pest tolerance such as genes for fungal disease control, herbicide tolerance such as genes conferring glyphosate tolerance, and genes for quality improvements such as environmental or stress tolerances, or any desirable changes in plant physiology, growth, development, morphology or plant product(s). For example, structural genes would include any gene that confers insect tolerance including but not limited to a Bacillus insect control protein gene as described in WO 99/31248, U.S. Pat. Nos. 5,689,052, 5,500,365 and 5,880,275, each of which are herein incorporated by reference in their entirety. In another embodiment, the structural gene can confer tolerance to the herbicide glyphosate as conferred by genes including, but not limited to Agrobacterium strain C′P4′ glyphosate resistant EPSPS gene (aroA:C′P4′) as described in U.S. Pat. No. 5,633,435, herein incorporated by reference in its entirety, or glyphosate oxidoreductase gene (GOX) as described in U.S. Pat. No. 5,463,175, herein incorporated by reference in its entirety.

Alternatively, the DNA coding sequences can affect these phenotypes by encoding a non-translatable RNA molecule that causes the targeted inhibition of expression of an endogenous gene, for example via antisense- or co-suppression-mediated mechanisms. The RNA could also be a catalytic RNA molecule (i.e., a ribozyme) engineered to cleave a desired endogenous mRNA product. Thus, any gene which produces a protein or mRNA which expresses a phenotype or morphology change of interest is useful for the practice of the present disclosure.

### E. Genetic Complements

In another aspect of the invention, the genetic complement of the sweetpotato plant line designated ‘P4’ or ‘R5’ is provided. The phrase “genetic complement” is used to refer to the aggregate of nucleotide sequences, the expression of which sequences defines the phenotype of, in the present case, an sweetpotato plant, or a cell or tissue of that plant. A genetic complement thus represents the genetic makeup of cell, tissue or plant, and a hybrid genetic complement represents the genetic makeup of a hybrid cell, tissue or plant. The invention thus provides sweetpotato (i.e. Ipomoea batatas) plant cells that have a genetic complement in accordance with the sweetpotato plant cells disclosed herein, and plants, seeds and polyploid plants containing such cells.

Plant genetic complements may be assessed by genetic marker profiles, and by the expression of phenotypic traits that are characteristic of the expression of the genetic complement, e.g., isozyme typing profiles. It is understood that line ‘P4’ or ‘R5’ could be identified by any of the many well-known techniques such as, for example, Simple Sequence Length Polymorphisms (SSLPs) (Williams et al., Nucleic Acids Res., 18:6531-6535, 1990), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs) (EP 534 858, specifically incorporated herein by reference in its entirety), and Single Nucleotide Polymorphisms (SNPs) (Wang et al., Science, 280:1077-1082, 1998).

In yet another aspect, the present invention provides hybrid genetic complements, as represented by sweetpotato plant cells, tissues, plants, and seeds, formed by the combination of a genetic complement of an sweetpotato plant of the invention with a genetic complement of the same or a different line. In another aspect, the present invention provides an sweetpotato plant regenerated from a tissue culture that comprises a hybrid genetic complement of this invention.

### F. Additional Traits

Additional traits can be introduced into the sweetpotato lines of the present invention. A non-limiting example of such a trait is a coding sequence that decreases RNA and/or protein levels. The decreased RNA and/or protein levels may be achieved through RNAi methods, such as those described in U.S. Pat. No. 6,506,559 to Fire and Mellow.

Another trait that may find use with the sweetpotato lines of the invention is a sequence that allows for site-specific recombination. Examples of such sequences include the FRT sequence, used with the FLP recombinase (Zhu and Sadowski, J. Biol. Chem., 270:23044-23054, 1995); and the LOX sequence, used with CRE recombinase (Sauer, Mol. Cell. Biol., 7:2087-2096, 1987). The recombinase genes can be encoded at any location within the genome of the sweetpotato plant, and are active in the hemizygous state.

It may also be desirable to make sweetpotato plants more tolerant to or more easily transformed with Agrobacterium tumefaciens. Expression of p53 and iap, two baculovirus cell-death suppressor genes, inhibited tissue necrosis and DNA cleavage. Additional targets can include plant-encoded proteins that interact with the Agrobacterium Vir genes; enzymes involved in plant cell wall formation; and histones, histone acetyltransferases and histone deacetylases (reviewed in Gelvin, Microbiology & Mol. Biol. Reviews, 67:16-37, 2003).

### G. Plants Comprising Non-Transgenic Mutations

In still yet another aspect, a plant of sweetpotato line ‘P4’ or ‘R5’, further comprising a non-transgenic mutation is provided. The phrase “non-transgenic mutation” is used herein to refer to a mutation that is naturally occurring (spontaneous), or induced by conventional methods (e.g. exposure of plants to radiation or mutagenic compounds), not including mutations made using recombinant DNA techniques. Various mutagenesis techniques have been developed and may be used by those of skill in the art to induce mutations in plants. Methods of mutagenesis may include, for example, exposure to irradiation, mutagenic compounds, extreme heat, or tissue culture conditions; long-term seed storage; and targeting induced local lesions in genomes (TILLING). In some embodiments, ionizing radiation may be produced by X-rays, gamma rays, neutrons, beta rays, or ultraviolet rays. Non-limiting examples of chemical mutagens include base analogues, antibiotics, alkylating agents, sodium azide, hydroxylamine, nitrous acid, methylnitrilsourea, and acridines. Methods of mutagenesis to modify, delete, or insert polynucleotides into the genomic DNA are well-known in the art.

In one aspect, improved sweetpotato lines may be created through mutation of the plant genome. In one embodiment, a plant of the sweetpotato line ‘P4’ or ‘R5’ may be subjected to a mutagenesis technique to create a population of mutant plants. Such mutant plants, for example, may comprise a mutation and otherwise comprise all of the physiological and morphological characteristics of sweetpotato line ‘P4’ or ‘R5’.

### H. Tissue Cultures and In Vitro Regeneration of Sweetpotato Plants

In another aspect, the invention relates to tissue cultures of the sweetpotato lines designated ‘P4’ and ‘R5’. As used herein, the term “tissue culture” indicates a composition comprising isolated cells of the same or a different type or a collection of such cells organized into parts of a plant. Exemplary types of tissue cultures are protoplasts, calli and plant cells that are intact in plants or parts of plants, such as embryos, meristems, cotyledons, pollen, leaves, anthers, roots, root tips, pistil, flower, florets, seed, stems, and the like. In a preferred embodiment, the tissue culture comprises cells derived from immature tissues of these plant parts. Means for preparing and maintaining plant tissue cultures are well known in the art (See, e.g. Panta, A. and Dodds, J. H., 1992. Tissue culture of Ipomoea batatas: micropropagation and maintenance. CIP; Arrigoni-Blank Mde F, et al. In vitro conservation of sweet potato genotypes. ScientificWorldJournal. 2014; 2014:208506.; and Behera S, et al. Biology and biotechnological aspect of sweet potato (Ipomoea batatas L.): a commercially important tuber crop. Planta. 2022; 256(2):40 incorporated herein by reference in their entirety).

In yet another aspect, compositions are provided comprising a cell of sweetpotato line designated ‘P4’ or ‘R5’ comprised in plant cell growth media. Plant cell growth media are well known to those of skill in the art. Plant cell growth media can provide adequate support for plant cells, including providing moisture and/or nutritional components.

### I. Processes of Crossing Sweetpotato Plants and the Sweetpotato Plants Produced by Such Crosses

The present invention provides processes of preparing novel sweetpotato plants and sweetpotato plants produced by such processes. In accordance with such a process, a first parent sweetpotato plant may be crossed with a second parent sweetpotato plant wherein at least one of the first and second sweetpotato plants is the sweetpotato line ‘P4’ or the sweetpotato line ‘R5’. One application of the process is in the production of F₁ hybrid plants. Another important aspect of this process is that it can be used for the development of novel sweetpotato lines (also known as cultivars and varieties). For example, the sweetpotato plant ‘P4’ or ‘R5’ could be crossed to any compatible second plant, and the resulting hybrid progeny could be vegetatively propagated or the hybrid progeny could be each selfed or further crossed for about 5 to 7 or more generations, thereby providing a large number of distinct lines. These lines could then be crossed with other lines and the resulting hybrid progeny analyzed for beneficial characteristics. In this way, novel lines conferring desirable characteristics could be identified. “Vegetative propagation” as used herein refers to any form of asexual reproduction occurring in plants in which a new plant grows from a fragment of the parent plant. Non-limiting examples of vegetative propagation methods include tissue culture and division.

### J. F₁ Hybrid Sweetpotato Plant and Seed Production

One beneficial use of the instant sweetpotato lines is in the production of hybrid seed. Any time the sweetpotato line ‘P4’ or ‘R5’ is crossed with another, different, sweetpotato plant, a first generation (F₁) sweetpotato hybrid plant is produced. As such, an F₁ hybrid sweetpotato plant can be produced by crossing ‘P4’ or ‘R5’ with any second, compatible sweetpotato plant. Numerous other sweetpotato plants can be used to produce a hybrid sweetpotato plant having either sweetpotato plant ‘P4’ or ‘R5’ as one parent.

The goal of the process of producing an F₁ hybrid is to manipulate the genetic complement of Ipomoea batatas to generate new combinations of genes that interact to yield new or improved traits (phenotypic characteristics).

Ipomoea batatas is a hexaploid (2n=6×=90) and has a large genome approximately 2-3 Gb in size. If the alleles are the same at a locus, there is said to be homozygosity. If they are different, there is said to be heterozygosity.

Hundreds of sweetpotato varieties are known to those of skill in the art, many one of which could be crossed with sweetpotato line ‘P4’ or ‘R5’ to produce a hybrid plant. For example, the US sweetpotato germplasm collection is maintained by the USDA, ARS, Plant Genetic Resources Conservation Unit (PGRCU) in Griffin, Georgia, United States. This genebank maintains a diverse collection of Ipomoea spp. and provides clonal propagules of sweetpotato that are maintained as in vitro cultures.

When the sweetpotato line ‘P4’ or ‘R5’ is crossed with another sweetpotato plant to yield a hybrid, it can serve as either the maternal or paternal plant. Depending on the seed production characteristics relative to a second parent in a hybrid cross, it may be desired to use one of the parental plants as the male or female parent. Seed coat characteristics can be preferable in one plant. Pollen can be shed better by one plant. Therefore, a decision to use one parent plant as a male or female may be made based on any such characteristics as is well known to those of skill in the art.

### K. Development of Sweetpotato Varieties

The development of new varieties using one or more starting varieties is well known in the art. In accordance with the invention, novel varieties may be created by crossing sweetpotato line ‘P4’ or ‘R5’ followed by vegetative propagation of selected plants. In certain embodiments, novel varieties may be created by crossing sweetpotato line ‘P4’ or ‘R5’ followed by multiple generations of breeding according to such well-known methods. New varieties may be created by crossing sweetpotato line ‘P4’ or ‘R5’ with any second plant. In selecting such a second plant to cross for the purpose of developing novel varieties, it may be desired to choose those plants that either themselves exhibit one or more selected desirable characteristics or exhibit the desired characteristic(s) when in hybrid combination. Examples of potentially desired characteristics include self- or cross-compatibility, flesh color, sweetpotato size, color patterning, foliage quality, shape and uniformity, maturity date, sweetpotato yield, seed germination rate, seedling vigor, pest and disease resistance (e.g. weevil resistance), herbicide tolerance, and adaptability for soil and climate conditions.

Once initial crosses have been made with sweetpotato line ‘P4’ or ‘R5’, vegetative propagation or inbreeding takes place to produce new varieties. Inbreeding requires manipulation by human breeders. Even in the extremely unlikely event inbreeding rather than crossbreeding occurred in natural Ipomoea batatas, achievement of complete inbreeding cannot be expected in nature due to well-known deleterious effects of homozygosity and the large number of generations the plant would have to breed in isolation. The reason for the breeder to create inbred plants is to have a known reservoir of genes whose gametic transmission is predictable.

The pedigree breeding method involves crossing two genotypes. Each genotype can have one or more desirable characteristics lacking in the other; or, each genotype can complement the other. If the two original parental genotypes do not provide all of the desired characteristics, other genotypes can be included in the breeding population. Superior plants that are the products of these crosses are selfed and selected in successive generations. Each succeeding generation becomes more homogeneous as a result of self-pollination and selection. Typically, this method of breeding involves five or more generations of selfing and selection: S₁→S₂; S₂→S₃; S₃→S₄; S₄→S₅, etc. After at least five generations, the inbred plant is considered genetically pure.

### EXAMPLES

### Example 1

### Evaluation of Weevil-Resistance Among Sweetpotato Germplasm Lines and Hybridization

From 2013 to 2016, more than 40 sweetpotato germplasm lines were evaluated, including many cultivars popularly grown in USA and three purple-fleshed ones, for their weevil resistance in the Gov Bill & Vara farm of PVAMU and nearby farms in Waller County. The weevil infection rate for various lines were semi-quantitatively estimated by sectioning sweetpotatoes from ˜50 plants each in at least triplicate rows in the field. As summarized in Table 5, all these lines except three could yield non-infected sweetpotatoes for only less than 25% of the total in plots that did not grow sweetpotato for at least three years but were not treated with insecticides. Two lines, Beauregard and SC 1149, could produce non-infected sweetpotatoes at about 25 to 50% of the total. The ‘Resisto’ cultivar (Jones et al. 1983) is the only sweetpotato line that could yield non-infected sweetpotatoes at 75 to 90%, or close to 100% of the total in plots without or with treatments of insecticides, respectively.

TABLE 5
Evaluation of Sweetpotato Germplasm for Weevil Resistance

| | | | Average rate of weevil | | |
|---|---|---|---|---|---|
| | | | infection (3-year trials¹) | | |
| Accession² | Name | Origin | 75%-100% | 50%-75% | 25%-10% |
| PI 296116 | Bnas51 | Philippines | + | | |
| PI 508509 | Norin 2 | Tiba, Japan | + | | |
| PI 508520 | Self-fertile No. 1 | China | + | | |
| PI 508524 | Mina | Puerto Rico, USA | + | | |
| PI 508534 | Batata Morada | Venezuela | + | | |
| PI 531122 | Jewel, Peru | Peru | + | | |
| PI 538300 | Papino | Peru | + | | |
| PI 538343 | Sunny | Puerto Rico, USA | + | | |
| PI 556934 | Cuitzeo | Honduras | + | | |
| PI 561255 | DLP 5296 | Guayas, Ecuador | + | | |
| PI 564126 | IB013 | Samoa | + | | |
| PI 564139 | L135 | Papua New Guinea | + | | |
| PI 566613 | Beauregard | USA | | + | |
| PI 566625 | Excel | USA | + | | |
| PI 566627 | Georgia Jet | USA | + | | |
| PI 566638 | Jewel, NC | USA | + | | |
| PI 566641 | Nancy Hall | USA | + | | |
| PI 566651 | ‘Resisto’ | USA | | | + |
| PI 566662 | Vardaman | USA | + | | |
| PI 573319 | Siale | Tonga | + | | |
| PI 595873 | Xushu-18 | China | + | | |
| PI 599369 | Haiti | Cuba | + | | |
| PI 599392 | GaoZi 1 | China | + | | |
| PI 633441 | Ayui | Uruguay | + | | |
| PI 633442 | LosCerrias1 | Uruguay | + | | |
| PI 634401 | SC1149-19 | SC, USA | | + | |
| PI 634444 | W-230 | USA | + | | |
| PI 636312 | Galona | Peru | + | | |
| PI 645581 | Bunch Porto | Porto Rico | + | | |
| | Rico | | | | |
| PI566619 | Centennial | USA | + | | |
| PI634376 | Garnet | USA | + | | |
| | Boniato | Cuba | + | | |
| | AsuF | In-house breeding | + | | |
| | | line | | | |
| | O'Henry | USA | + | | |
| | Covington | USA | + | | |
| | Murasaki-1 | USA | + | | |
| | Bonita | USA | + | | |
| | | | + | | |
| PI 573297 | Mayama (Purple) | Burma | + | | |
| | ‘CH-Purple’ | USA | + | | |
| | Japanese Purple | Japan | + | | |

¹The evaluations were conducted on experimental plots without prior sweetpotato cultivation for at least two years and without soil treatment with insecticide in the Gov Bill and Vara Farm and Ranch of Prairie View A&M University.
²All evaluated cultivars with an accession number were obtained from the USDA-ARS Plant Genetic Resources Conservation Unit, Griffin, GA (https://npgsweb.ars-grin.gov/gringlobal/site.aspx?id=22). Another six cultivars, including Boniato, Japanese purple, O'Henry, Murasaki-1, Covington and Bonita were originally from a commercial source (https://www.sweetpotatoplant.com). The ‘CH-Purple’ and CH-vegie lines were derived from locally grown unknown cultivars. The AsuF line was an in-house breeding line.

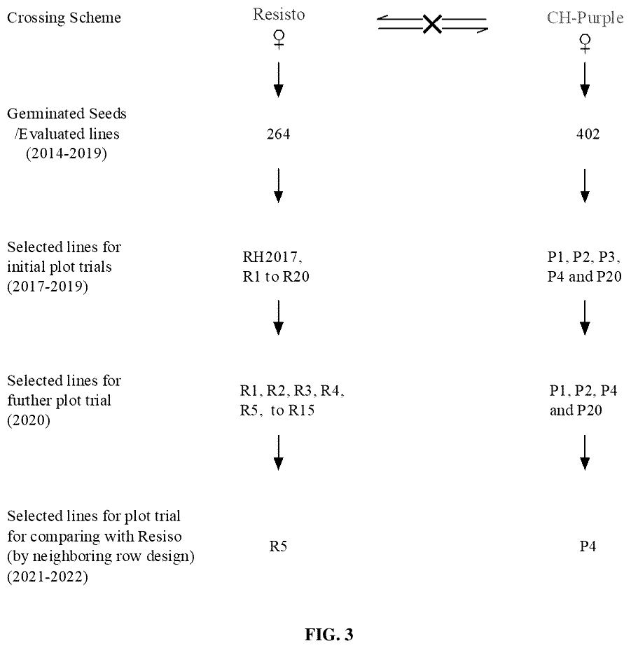

Among the three purple-fleshed sweetpotato germplasm lines, the ‘CH-Purple’ and PI573297 were cross-compatible with ‘Resisto’. The ‘CH-Purple’ was originally sprouted from sweetpotatoes sold in a local farmer's market, and thus without a cultivar identity. Four rounds of hybridization were performed between the two purple-fleshed lines and ‘Resisto’ from 2016 to 2019 through natural pollination, and harvested seeds from both the purple-fleshed lines and ‘Resisto’. About 30% of the germinated lines mothered by the two purple-fleshed lines produced purple-fleshed sweetpotatoes, while less than 5% of those mothered by ‘Resisto’ yielded purple-fleshed sweetpotatoes. No lines were selected from crosses between PI573297 and ‘Resisto’. FIG. 3 summarizes the crossing scheme between the ‘CH-Purple’ and ‘Resisto’, and the selection process for the two new purple-fleshed sweetpotato lines, ‘P4’ and ‘R5’. As illustrated, the ‘P4’ and the ‘R5’ purple-fleshed lines were selected among initially 402 germinated ‘CH-Purple’-mothered and 264 ‘Resisto’-mothered hybrids (2017 to 2019) through four or three rounds of plot evaluation and trials, respectively, including one for comparing with ‘Resisto’ in triplicated neighboring row design. The introgression process was continued by backcrossing the ‘P4’ line to ‘Resisto’ and generating a very large backcross population for further selection. It was found that the ‘P4’ line displayed improved weevil resistance, and yielded sweetpotatoes having features for processing and vegetables uses. It was also found that the ‘R5’ purple-fleshed line has additional, highly desirable features such a self-compatibility.

### Example 2

### Substantially Improved Weevil Resistance and Additional Distinguishing Characteristics of Sweetpotato Lines ‘P4’ and ‘R5’

The ‘P4’ line has a very robust growing pattern, improved weevil resistance, and average disease resistance, and produces sweetpotatoes in good shape and sizes. It has a day-neutral photoperiod, producing copious flowers under cool temperature in pots and in field. It has a wider cross-compatibility, capable of crossing to the parental lines, many F1 siblings and many germplasm lines. In particular, the young leaves and shoots of the ‘P4’ line contains a high anthocyanin content as indicated by the purple color of young leaves, and not as stingy and bitter flavor when cooked as most of the breeding lines and cultivars used.

The lesser stinginess and bitterness of the young leaves and shoots of the ‘P4’ line are due to a low content of tannins, phytates, oxalates, and lectins, which is most likely the reason why they are much less resistant against leaf-eater insects. However, this feature made the line an excellent candidate for using the young leaves and shoots as vegetables. Additionally, the ‘P4’ line produced sweetpotatoes having much less reduction of anthocyanin content as compared to those from the maternal ‘CH-Purple’ line and the other purple-fleshed hybrids evaluated during the breeding process, and a high dry-matter content. Thus, ‘P4’ may be a good candidate for vegetable use and producing sweetpotatoes for processing into flours or other food ingredients for nutrition fortification and natural food coloring.

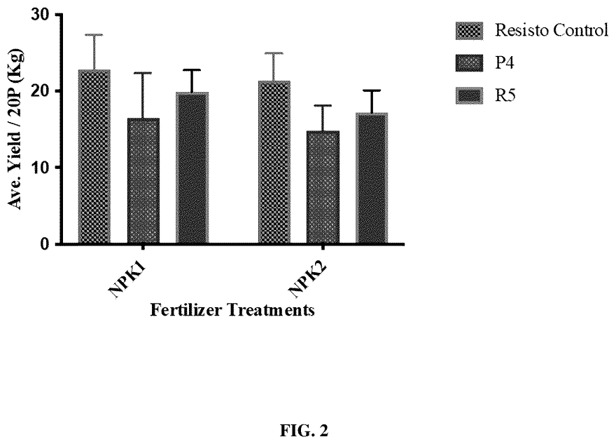

The ‘P4’ and ‘R5’ lines having substantially improved weevil resistance could yield mostly weevil-free sweetpotatoes (>99%) when grown under an integrated pest management (IPM). The IPM includes at least two-year crop rotation, soil treatment before making planting beds and foliar and soil-surface spray around 60 days after transplanting with Chlorpyrifos 4E (State-registered) at a rate of 6 to 7 pt./ac, and intermittent foliar sprays with Carbaryl 4L during the growing period (every three weeks or so) at a rate of 2 qtrs./ac. Under such an IPM, we compared the fresh sweetpotato yields of the ‘P4’ and ‘R5’ to the parental ‘Resisto’ using a triplicated side-by-side double-row design during 2021 and 2022 growing season, and summarized the results in FIG. 2. On average, the fresh sweetpotato yields of the ‘P4’ and ‘R5’ were about 69-73% and 80-87% of those of ‘Resisto’. Both lines have large enough yields for potentially profitable commercial cultivations. Since the breeding site at the Farm of Prairie View A&M University is in the weevil-quarantined area, we made aseptic tissue-cultured germplasms of both lines, transferred the aseptic germplasms to the Texas A&M AgriLife Center at Dallas, and regenerated the two lines (Go) in greenhouse at the center in 2022. Variety trials will be conducted during the 2023 growing season to evaluate their agronomical and yield performances as compared to commercial varieties.

All of the compositions and methods disclosed and claimed herein can be made and executed without undue experimentation in light of the present disclosure. While the compositions and methods of this invention have been described in terms of the foregoing illustrative embodiments, it will be apparent to those of skill in the art that variations, changes, modifications, and alterations may be applied to the composition, methods, and in the steps or in the sequence of steps of the methods described herein, without departing from the true concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

The references cited herein, to the extent that they provide exemplary procedural or other details supplementary to those set forth herein, are specifically incorporated herein by reference.

1. A sweetpotato plant of line ‘P4’, a representative sample of plant tissue of said line having been deposited under NCMA Accession No. ______.

2. A sweetpotato plant of line ‘R5’, a representative sample of plant tissue of said line having been deposited under NCMA Accession No. ______.

3. A seed of the plant of claim 1.

4. A plant part of the plant of claim 1, wherein said plant part comprises at least one cell of said plant.

5. The plant part of claim 3, defined as a flower, pollen, a leaf, an ovule, an embryo, a cutting, an axillary bud, a stem, a root, or a seed.

6. A sweetpotato plant having all of the physiological and morphological characteristics of the plant of claim 1.

7. A tissue culture of regenerable cells of the plant of claim 1.

8. A sweetpotato plant regenerated from the tissue culture of claim 7.

9. A method for producing a first generation progeny sweetpotato seed, the method comprising crossing the plant of claim 1 with itself or a second sweetpotato plant and harvesting the resultant sweetpotato seed.

10. The method of claim 9, wherein the second sweetpotato plant is:
a plant of sweetpotato line ‘P4’, wherein representative plant tissue of said sweetpotato line have been deposited under NCMA Accession No. ______
a plant of sweetpotato line ‘R5’, wherein representative plant tissue of said sweetpotato live have been deposited under NCMA Accession No. ______.

11. A first generation progeny sweetpotato seed produced by the method of claim 9; wherein the first generation progeny sweetpotato plant produced from said seed has all of the physiological and morphological characteristics of a plant of sweetpotato line ‘P4’.

12. A sweetpotato plant produced by growing the seed of claim 11; wherein the plant produced has all of the physiological and morphological characteristics of a plant of sweetpotato line ‘P4’.

13. A method of vegetatively propagating the plant of claim 1, the method comprising the steps of:
(a) collecting tissue capable of being propagated from the plant of claim 1; and
(b) propagating a plant from said tissue.

14. A method of modifying an sweetpotato plant, wherein the method comprises introducing a transgene or a single locus conversion into the plant of claim 1.

15. The sweetpotato plant produced by the method of claim 14; wherein the sweetpotato plant produced otherwise comprises all of the physiological and morphological characteristics of a plant of sweetpotato line ‘P4’.

16. The plant of claim 15, wherein the transgene or single locus comprises a nucleic acid sequence that enables site-specific genetic recombination or confers a trait selected from the group consisting of male sterility, herbicide tolerance, insect resistance, pest resistance, disease resistance, improved digestibility, improved energy content, improved forage or seed yield, improved winterhardiness, improved nitrogen fixation, modified fatty acid metabolism, abiotic stress resistance, flowering time, altered seed amino acid composition, and modified carbohydrate metabolism.

17. A seed that produces the plant of claim 15; wherein the seed otherwise comprises all of the physiological and morphological characteristics of a seed of said sweetpotato line ‘P4’.

18. A method of introducing a single-locus conversion into the plant of claim 1, the method comprising:
(a) crossing said plant with a second sweetpotato plant to produce a first generation of progeny plants, wherein the second sweetpotato plant comprises the single locus; and
(b) selecting a progeny plant that comprises the single locus.

19. The method of claim 18, wherein the single locus comprises a transgene.

20. A sweetpotato plant produced by the method of claim 18; wherein the sweetpotato plant produced otherwise comprises all of the physiological and morphological characteristics of a plant of sweetpotato line ‘P4’.

21. A seed that produces the plant of claim 20.

22. A method for introducing a transgene or a single locus conversion into a population of sweetpotato plants, the method comprising the steps of:
(a) modifying the plant of claim 1 by introducing a transgene or a single locus conversion; and
(b) crossing the modified sweetpotato plant of step (a) with a population of sweetpotato plants to produce a population of progeny plants, wherein at least a progeny plant comprises the transgene or single locus conversion.

23. The method of claim 22, further comprising the step of:
(c) applying a selection technique to the population produced in step (b) to select said progeny plants that comprise the transgene or single locus conversion.

24. A method of producing a commodity plant product, the method comprising producing the commodity plant product from the plant of claim 1 or plant part thereof.

25. The method of claim 24, wherein the commodity plant product is selected from a group consisting of tuberous root, flour, starch, juice, bread, and pectin.

26. A commodity plant product produced by the method of claim 24, wherein the commodity plant product comprises at least one cell of sweetpotato line ‘P4’.

27. A method of plant breeding comprising applying plant breeding techniques to a plant according to claim 1.

28. The method of claim 27:
defined as comprising producing a sweetpotato line ‘P4’-derived sweetpotato plant; or
wherein said plant breeding techniques comprise recurrent selection, mass selection, hybridization, open-pollination, backcrossing, modified backcrossing, pedigree breeding, mutation breeding, or marker assisted selection.

29. (canceled)

30. The method of claim 28, further defined as comprising selecting a sweetpotato line ‘P4’-derived sweetpotato plant that comprises:
a purple skin trait found in sweetpotato line ‘P4’;
a purple flesh trait found in sweetpotato line ‘P4’; or
a weevil resistance trait found in sweetpotato line ‘P4’.

31. (canceled)
